SEQUENCE LISTING (401 sequences)

SEQ ID NO: 1 - DNA, 21 bp, Artificial Sequence
  Description of Artificial Sequence Synthetic oligonucleotide
ggaccagugugcuucgucutt

SEQ ID NO: 2 - DNA, 21 bp, Artificial Sequence
  Description of Artificial Sequence Synthetic oligonucleotide
agacgaagcacacuggucctt

SEQ ID NO: 3 - DNA, 21 bp, Artificial Sequence
  Description of Artificial Sequence Synthetic oligonucleotide
ggacccuucauuagaucgctt

SEQ ID NO: 4 - DNA, 21 bp, Artificial Sequence
  Description of Artificial Sequence Synthetic oligonucleotide
gcgaucuaaugaagggucctt

SEQ ID NO: 5 - DNA, 21 bp, Artificial Sequence
  Description of Artificial Sequence Synthetic oligonucleotide
accucauauaguagugaggtt

SEQ ID NO: 6 - DNA, 21 bp, Artificial Sequence
  Description of Artificial Sequence Synthetic oligonucleotide
ccucacuacuauaugaggutt

SEQ ID NO: 7 - DNA, 21 bp, Artificial Sequence
  Description of Artificial Sequence Synthetic oligonucleotide
gaacguucgugguuccgugtt

SEQ ID NO: 8 - DNA, 21 bp, Artificial Sequence
  Description of Artificial Sequence Synthetic oligonucleotide
cacggaaccacgaacguuctt

SEQ ID NO: 9 - DNA, 21 bp, Artificial Sequence
  Description of Artificial Sequence Synthetic oligonucleotide
aauuccucgagauaggccgtt

SEQ ID NO: 10 - DNA, 21 bp, Artificial Sequence
  Description of Artificial Sequence Synthetic oligonucleotide
cggccuaucucgaggaauutt

SEQ ID NO: 11 - DNA, 21 bp, Artificial Sequence
  Description of Artificial Sequence Synthetic oligonucleotide
uuccucgagauaggccguutt

SEQ ID NO: 12 - DNA, 21 bp, Artificial Sequence
  Description of Artificial Sequence Synthetic oligonucleotide
aacggccuaucucgaggaatt

SEQ ID NO: 13 - DNA, 21 bp, Artificial Sequence
  Description of Artificial Sequence Synthetic oligonucleotide
gaacaauugcgagaacuautt

SEQ ID NO: 14 - DNA, 21 bp, Artificial Sequence
  Description of Artificial Sequence Synthetic oligonucleotide
auaguucucgcaauuguuctt

SEQ ID NO: 15 - DNA, 21 bp, Artificial Sequence
  Description of Artificial Sequence Synthetic oligonucleotide
gagaagaacguucgugguutt

SEQ ID NO: 16 - DNA, 21 bp, Artificial Sequence
  Description of Artificial Sequence Synthetic oligonucleotide
aaccacgaacguucuucuctt

SEQ ID NO: 17 - DNA, 21 bp, Artificial Sequence
  Description of Artificial Sequence Synthetic oligonucleotide
aagaacguucgugguuccgtt

SEQ ID NO: 18 - DNA, 21 bp, Artificial Sequence
  Description of Artificial Sequence Synthetic oligonucleotide
cggaaccacgaacguucuutt

SEQ ID NO: 19 - DNA, 21 bp, Artificial Sequence
  Description of Artificial Sequence Synthetic oligonucleotide
uugcucgacgauguuccautt

SEQ ID NO: 20 - DNA, 21 bp, Artificial Sequence
  Description of Artificial Sequence Synthetic oligonucleotide
auggaacaucgucgagcaatt

SEQ ID NO: 21 - DNA, 21 bp, Artificial Sequence
  Description of Artificial Sequence Synthetic oligonucleotide
aaacauuaucgcaacucagtt

SEQ ID NO: 22 - DNA, 21 bp, Artificial Sequence
  Description of Artificial Sequence Synthetic oligonucleotide
cugaguugcgauaauguuutt

SEQ ID NO: 23 - DNA, 21 bp, Artificial Sequence
  Description of Artificial Sequence Synthetic oligonucleotide
aacuguaauguuacgucautt

SEQ ID NO: 24 - DNA, 21 bp, Artificial Sequence
  Description of Artificial Sequence Synthetic oligonucleotide
augacguaacauuacaguutt

SEQ ID NO: 25 - DNA, 21 bp, Artificial Sequence
  Description of Artificial Sequence Synthetic oligonucleotide
gacccuucauuagaucgcctt

SEQ ID NO: 26 - DNA, 21 bp, Artificial Sequence
  Description of Artificial Sequence Synthetic oligonucleotide
ggcgaucuaaugaaggguctt

SEQ ID NO: 27 - DNA, 21 bp, Artificial Sequence
  Description of Artificial Sequence Synthetic oligonucleotide
guccacggcgagcggucuutt

SEQ ID NO: 28 - DNA, 21 bp, Artificial Sequence
  Description of Artificial Sequence Synthetic oligonucleotide
aagaccgcucgccguggactt

SEQ ID NO: 29 - DNA, 21 bp, Artificial Sequence
  Description of Artificial Sequence Synthetic oligonucleotide
cgagauaggccguuuguautt

SEQ ID NO: 30 - DNA, 21 bp, Artificial Sequence
  Description of Artificial Sequence Synthetic oligonucleotide
auacaaacggccuaucucgtt

SEQ ID NO: 31 - DNA, 21 bp, Artificial Sequence
  Description of Artificial Sequence Synthetic oligonucleotide
cuuacagcauugcggauuatt

SEQ ID NO: 32 - DNA, 21 bp, Artificial Sequence
  Description of Artificial Sequence Synthetic oligonucleotide
uaauccgcaaugcuguaagtt

SEQ ID NO: 33 - DNA, 21 bp, Artificial Sequence
  Description of Artificial Sequence Synthetic oligonucleotide
gcuuacagcauugcggauutt

SEQ ID NO: 34 - DNA, 21 bp, Artificial Sequence
  Description of Artificial Sequence Synthetic oligonucleotide
aauccgcaaugcuguaagctt

SEQ ID NO: 35 - DNA, 21 bp, Artificial Sequence
  Description of Artificial Sequence Synthetic oligonucleotide
uccucgagauaggccguuutt

SEQ ID NO: 36 - DNA, 21 bp, Artificial Sequence
  Description of Artificial Sequence Synthetic oligonucleotide
aaacggccuaucucgaggatt

SEQ ID NO: 37 - DNA, 21 bp, Artificial Sequence
  Description of Artificial Sequence Synthetic oligonucleotide
cucgagauaggccguuugutt

SEQ ID NO: 38 - DNA, 21 bp, Artificial Sequence
  Description of Artificial Sequence Synthetic oligonucleotide
acaaacggccuaucucgagtt

SEQ ID NO: 39 - DNA, 21 bp, Artificial Sequence
  Description of Artificial Sequence Synthetic oligonucleotide
acccuucauuagaucgccctt

SEQ ID NO: 40 - DNA, 21 bp, Artificial Sequence
  Description of Artificial Sequence Synthetic oligonucleotide
gggcgaucuaaugaagggutt

SEQ ID NO: 41 - DNA, 21 bp, Artificial Sequence
  Description of Artificial Sequence Synthetic oligonucleotide
uucagaggguacuacguugtt

SEQ ID NO: 42 - DNA, 21 bp, Artificial Sequence
  Description of Artificial Sequence Synthetic oligonucleotide
caacguaguacccucugaatt

SEQ ID NO: 43 - DNA, 21 bp, Artificial Sequence
  Description of Artificial Sequence Synthetic oligonucleotide
ucagaggguacuacguugatt

SEQ ID NO: 44 - DNA, 21 bp, Artificial Sequence
  Description of Artificial Sequence Synthetic oligonucleotide
ucaacguaguacccucugatt

SEQ ID NO: 45 - DNA, 21 bp, Artificial Sequence
  Description of Artificial Sequence Synthetic oligonucleotide
cagaggguacuacguugaatt

SEQ ID NO: 46 - DNA, 21 bp, Artificial Sequence
  Description of Artificial Sequence Synthetic oligonucleotide
uucaacguaguacccucugtt

SEQ ID NO: 47 - DNA, 21 bp, Artificial Sequence
  Description of Artificial Sequence Synthetic oligonucleotide
agaagaacguucgugguuctt

SEQ ID NO: 48 - DNA, 21 bp, Artificial Sequence
  Description of Artificial Sequence Synthetic oligonucleotide
gaaccacgaacguucuucutt

SEQ ID NO: 49 - DNA, 21 bp, Artificial Sequence
  Description of Artificial Sequence Synthetic oligonucleotide
acguucgugguuccgugagtt

SEQ ID NO: 50 - DNA, 21 bp, Artificial Sequence
  Description of Artificial Sequence Synthetic oligonucleotide
cucacggaaccacgaacgutt

SEQ ID NO: 51 - DNA, 21 bp, Artificial Sequence
  Description of Artificial Sequence Synthetic oligonucleotide
aaacuguaauguuacgucatt

SEQ ID NO: 52 - DNA, 21 bp, Artificial Sequence
  Description of Artificial Sequence Synthetic oligonucleotide
ugacguaacauuacaguuutt

SEQ ID NO: 53 - DNA, 21 bp, Artificial Sequence
  Description of Artificial Sequence Synthetic oligonucleotide
cuguauugcagacuuaggatt

SEQ ID NO: 54 - DNA, 21 bp, Artificial Sequence
  Description of Artificial Sequence Synthetic oligonucleotide
uccuaagucugcaauacagtt

SEQ ID NO: 55 - DNA, 21 bp, Artificial Sequence
  Description of Artificial Sequence Synthetic oligonucleotide
uuacuccugguuaguacautt

SEQ ID NO: 56 - DNA, 21 bp, Artificial Sequence
  Description of Artificial Sequence Synthetic oligonucleotide
auguacuaaccaggaguaatt

SEQ ID NO: 57 - DNA, 21 bp, Artificial Sequence
  Description of Artificial Sequence Synthetic oligonucleotide
ugaccucauauaguagugatt

SEQ ID NO: 58 - DNA, 21 bp, Artificial Sequence
  Description of Artificial Sequence Synthetic oligonucleotide
ucacuacuauaugaggucatt

SEQ ID NO: 59 - DNA, 21 bp, Artificial Sequence
  Description of Artificial Sequence Synthetic oligonucleotide
gguacuacguugaaagacutt

SEQ ID NO: 60 - DNA, 21 bp, Artificial Sequence
  Description of Artificial Sequence Synthetic oligonucleotide
agucuuucaacguaguacctt

SEQ ID NO: 61 - DNA, 21 bp, Artificial Sequence
  Description of Artificial Sequence Synthetic oligonucleotide
cauuaucgcaacucagucatt

SEQ ID NO: 62 - DNA, 21 bp, Artificial Sequence
  Description of Artificial Sequence Synthetic oligonucleotide
ugacugaguugcgauaaugtt

SEQ ID NO: 63 - DNA, 21 bp, Artificial Sequence
  Description of Artificial Sequence Synthetic oligonucleotide
gcgagaacuauuguguuactt

SEQ ID NO: 64 - DNA, 21 bp, Artificial Sequence
  Description of Artificial Sequence Synthetic oligonucleotide
guaacacaauaguucucgctt

SEQ ID NO: 65 - DNA, 21 bp, Artificial Sequence
  Description of Artificial Sequence Synthetic oligonucleotide
cgacggcguuacaguguuutt

SEQ ID NO: 66 - DNA, 21 bp, Artificial Sequence
  Description of Artificial Sequence Synthetic oligonucleotide
aaacacuguaacgccgucgtt

SEQ ID NO: 67 - DNA, 21 bp, Artificial Sequence
  Description of Artificial Sequence Synthetic oligonucleotide
agaacguucgugguuccgutt

SEQ ID NO: 68 - DNA, 21 bp, Artificial Sequence
  Description of Artificial Sequence Synthetic oligonucleotide
acggaaccacgaacguucutt

SEQ ID NO: 69 - DNA, 21 bp, Artificial Sequence
  Description of Artificial Sequence Synthetic oligonucleotide
ccucgagauaggccguuugtt

SEQ ID NO: 70 - DNA, 21 bp, Artificial Sequence
  Description of Artificial Sequence Synthetic oligonucleotide
caaacggccuaucucgaggtt

SEQ ID NO: 71 - DNA, 21 bp, Artificial Sequence
  Description of Artificial Sequence Synthetic oligonucleotide
gaucgcccuuuuauuucagtt

SEQ ID NO: 72 - DNA, 21 bp, Artificial Sequence
  Description of Artificial Sequence Synthetic oligonucleotide
cugaaauaaaagggcgauctt

SEQ ID NO: 73 - DNA, 21 bp, Artificial Sequence
  Description of Artificial Sequence Synthetic oligonucleotide
ucgcaacucagucaacaggtt

SEQ ID NO: 74 - DNA, 21 bp, Artificial Sequence
  Description of Artificial Sequence Synthetic oligonucleotide
ccuguugacugaguugcgatt

SEQ ID NO: 75 - DNA, 21 bp, Artificial Sequence
  Description of Artificial Sequence Synthetic oligonucleotide
acuaugaacgcuucuuucctt

SEQ ID NO: 76 - DNA, 21 bp, Artificial Sequence
  Description of Artificial Sequence Synthetic oligonucleotide
ggaaagaagcguucauagutt

SEQ ID NO: 77 - DNA, 21 bp, Artificial Sequence
  Description of Artificial Sequence Synthetic oligonucleotide
guacuauaccaguaagugctt

SEQ ID NO: 78 - DNA, 21 bp, Artificial Sequence
  Description of Artificial Sequence Synthetic oligonucleotide
gcacuuacugguauaguactt

SEQ ID NO: 79 - DNA, 21 bp, Artificial Sequence
  Description of Artificial Sequence Synthetic oligonucleotide
aauugacuuaauuccucgatt

SEQ ID NO: 80 - DNA, 21 bp, Artificial Sequence
  Description of Artificial Sequence Synthetic oligonucleotide
ucgaggaauuaagucaauutt

SEQ ID NO: 81 - DNA, 21 bp, Artificial Sequence
  Description of Artificial Sequence Synthetic oligonucleotide
uugacuuaauuccucgagatt

SEQ ID NO: 82 - DNA, 21 bp, Artificial Sequence
  Description of Artificial Sequence Synthetic oligonucleotide
ucucgaggaauuaagucaatt

SEQ ID NO: 83 - DNA, 21 bp, Artificial Sequence
  Description of Artificial Sequence Synthetic oligonucleotide
cugggucugugacuacaactt

SEQ ID NO: 84 - DNA, 21 bp, Artificial Sequence
  Description of Artificial Sequence Synthetic oligonucleotide
guuguagucacagacccagtt

SEQ ID NO: 85 - DNA, 21 bp, Artificial Sequence
  Description of Artificial Sequence Synthetic oligonucleotide
cugcaaucaggaccauugctt

SEQ ID NO: 86 - DNA, 21 bp, Artificial Sequence
  Description of Artificial Sequence Synthetic oligonucleotide
gcaaugguccugauugcagtt

SEQ ID NO: 87 - DNA, 21 bp, Artificial Sequence
  Description of Artificial Sequence Synthetic oligonucleotide
gaccagugugcuucgucugtt

SEQ ID NO: 88 - DNA, 21 bp, Artificial Sequence
  Description of Artificial Sequence Synthetic oligonucleotide
cagacgaagcacacugguctt

SEQ ID NO: 89 - DNA, 21 bp, Artificial Sequence
  Description of Artificial Sequence Synthetic oligonucleotide
cuauaucugccacaaccgctt

SEQ ID NO: 90 - DNA, 21 bp, Artificial Sequence
  Description of Artificial Sequence Synthetic oligonucleotide
gcgguuguggcagauauagtt

SEQ ID NO: 91 - DNA, 21 bp, Artificial Sequence
  Description of Artificial Sequence Synthetic oligonucleotide
aaccgcacugucauucacctt

SEQ ID NO: 92 - DNA, 21 bp, Artificial Sequence
  Description of Artificial Sequence Synthetic oligonucleotide
ggugaaugacagugcgguutt

SEQ ID NO: 93 - DNA, 21 bp, Artificial Sequence
  Description of Artificial Sequence Synthetic oligonucleotide
cauucaccaucgagugccatt

SEQ ID NO: 94 - DNA, 21 bp, Artificial Sequence
  Description of Artificial Sequence Synthetic oligonucleotide
uggcacucgauggugaaugtt

SEQ ID NO: 95 - DNA, 21 bp, Artificial Sequence
  Description of Artificial Sequence Synthetic oligonucleotide
agaggguacuacguugaaatt

SEQ ID NO: 96 - DNA, 21 bp, Artificial Sequence
  Description of Artificial Sequence Synthetic oligonucleotide
uuucaacguaguacccucutt

SEQ ID NO: 97 - DNA, 21 bp, Artificial Sequence
  Description of Artificial Sequence Synthetic oligonucleotide
auuuaugauaugacaacgutt

SEQ ID NO: 98 - DNA, 21 bp, Artificial Sequence
  Description of Artificial Sequence Synthetic oligonucleotide
acguugucauaucauaaautt

SEQ ID NO: 99 - DNA, 21 bp, Artificial Sequence
  Description of Artificial Sequence Synthetic oligonucleotide
cauuggcaaaggucgauuutt

SEQ ID NO: 100 - DNA, 21 bp, Artificial Sequence
  Description of Artificial Sequence Synthetic oligonucleotide
aaaucgaccuuugccaaugtt

SEQ ID NO: 101 - DNA, 21 bp, Artificial Sequence
  Description of Artificial Sequence Synthetic oligonucleotide
ugguuggugucagauuauctt

SEQ ID NO: 102 - DNA, 21 bp, Artificial Sequence
  Description of Artificial Sequence Synthetic oligonucleotide
gauaaucugacaccaaccatt

SEQ ID NO: 103 - DNA, 21 bp, Artificial Sequence
  Description of Artificial Sequence Synthetic oligonucleotide
gguuggugucagauuaucatt

SEQ ID NO: 104 - DNA, 21 bp, Artificial Sequence
  Description of Artificial Sequence Synthetic oligonucleotide
ugauaaucugacaccaacctt

SEQ ID NO: 105 - DNA, 21 bp, Artificial Sequence
  Description of Artificial Sequence Synthetic oligonucleotide
uuaucaugagcauggaucctt

SEQ ID NO: 106 - DNA, 21 bp, Artificial Sequence
  Description of Artificial Sequence Synthetic oligonucleotide
ggauccaugcucaugauaatt

SEQ ID NO: 107 - DNA, 21 bp, Artificial Sequence
  Description of Artificial Sequence Synthetic oligonucleotide
agcauggaucccuuuuugatt

SEQ ID NO: 108 - DNA, 21 bp, Artificial Sequence
  Description of Artificial Sequence Synthetic oligonucleotide
ucaaaaagggauccaugcutt

SEQ ID NO: 109 - DNA, 21 bp, Artificial Sequence
  Description of Artificial Sequence Synthetic oligonucleotide
caaugggcuuaguauucugtt

SEQ ID NO: 110 - DNA, 21 bp, Artificial Sequence
  Description of Artificial Sequence Synthetic oligonucleotide
cagaauacuaagcccauugtt

SEQ ID NO: 111 - DNA, 21 bp, Artificial Sequence
  Description of Artificial Sequence Synthetic oligonucleotide
cugggaaauugcucgacgatt

SEQ ID NO: 112 - DNA, 21 bp, Artificial Sequence
  Description of Artificial Sequence Synthetic oligonucleotide
ucgucgagcaauuucccagtt

SEQ ID NO: 113 - DNA, 21 bp, Artificial Sequence
  Description of Artificial Sequence Synthetic oligonucleotide
cucgacgauguuccauuggtt

SEQ ID NO: 114 - DNA, 21 bp, Artificial Sequence
  Description of Artificial Sequence Synthetic oligonucleotide
ccaauggaacaucgucgagtt

SEQ ID NO: 115 - DNA, 21 bp, Artificial Sequence
  Description of Artificial Sequence Synthetic oligonucleotide
ucgacgauguuccauuggutt

SEQ ID NO: 116 - DNA, 21 bp, Artificial Sequence
  Description of Artificial Sequence Synthetic oligonucleotide
accaauggaacaucgucgatt

SEQ ID NO: 117 - DNA, 21 bp, Artificial Sequence
  Description of Artificial Sequence Synthetic oligonucleotide
cgacgauguuccauuggugtt

SEQ ID NO: 118 - DNA, 21 bp, Artificial Sequence
  Description of Artificial Sequence Synthetic oligonucleotide
caccaauggaacaucgucgtt

SEQ ID NO: 119 - DNA, 21 bp, Artificial Sequence
  Description of Artificial Sequence Synthetic oligonucleotide
gacgauguuccauugguggtt

SEQ ID NO: 120 - DNA, 21 bp, Artificial Sequence
  Description of Artificial Sequence Synthetic oligonucleotide
ccaccaauggaacaucguctt

SEQ ID NO: 121 - DNA, 21 bp, Artificial Sequence
  Description of Artificial Sequence Synthetic oligonucleotide
gaaaacauuaucgcaacuctt

SEQ ID NO: 122 - DNA, 21 bp, Artificial Sequence
  Description of Artificial Sequence Synthetic oligonucleotide
gaguugcgauaauguuuuctt

SEQ ID NO: 123 - DNA, 21 bp, Artificial Sequence
  Description of Artificial Sequence Synthetic oligonucleotide
aaaacauuaucgcaacucatt

SEQ ID NO: 124 - DNA, 21 bp, Artificial Sequence
  Description of Artificial Sequence Synthetic oligonucleotide
ugaguugcgauaauguuuutt

SEQ ID NO: 125 - DNA, 21 bp, Artificial Sequence
  Description of Artificial Sequence Synthetic oligonucleotide
uaucgcaacucagucaacatt

SEQ ID NO: 126 - DNA, 21 bp, Artificial Sequence
  Description of Artificial Sequence Synthetic oligonucleotide
uguugacugaguugcgauatt

SEQ ID NO: 127 - DNA, 21 bp, Artificial Sequence
  Description of Artificial Sequence Synthetic oligonucleotide
cuacagcuuugccugaacutt

SEQ ID NO: 128 - DNA, 21 bp, Artificial Sequence
  Description of Artificial Sequence Synthetic oligonucleotide
aguucaggcaaagcuguagtt

SEQ ID NO: 129 - DNA, 21 bp, Artificial Sequence
  Description of Artificial Sequence Synthetic oligonucleotide
uucugugcacuaugaacgctt

SEQ ID NO: 130 - DNA, 21 bp, Artificial Sequence
  Description of Artificial Sequence Synthetic oligonucleotide
gcguucauagugcacagaatt

SEQ ID NO: 131 - DNA, 21 bp, Artificial Sequence
  Description of Artificial Sequence Synthetic oligonucleotide
ugcacuaugaacgcuucuutt

SEQ ID NO: 132 - DNA, 21 bp, Artificial Sequence
  Description of Artificial Sequence Synthetic oligonucleotide
aagaagcguucauagugcatt

SEQ ID NO: 133 - DNA, 21 bp, Artificial Sequence
  Description of Artificial Sequence Synthetic oligonucleotide
ugauuuacuccugguuagutt

SEQ ID NO: 134 - DNA, 21 bp, Artificial Sequence
  Description of Artificial Sequence Synthetic oligonucleotide
acuaaccaggaguaaaucatt

SEQ ID NO: 135 - DNA, 21 bp, Artificial Sequence
  Description of Artificial Sequence Synthetic oligonucleotide
uuuacuccugguuaguacatt

SEQ ID NO: 136 - DNA, 21 bp, Artificial Sequence
  Description of Artificial Sequence Synthetic oligonucleotide
uguacuaaccaggaguaaatt

SEQ ID NO: 137 - DNA, 21 bp, Artificial Sequence
  Description of Artificial Sequence Synthetic oligonucleotide
ccugguuaguacauucucatt

SEQ ID NO: 138 - DNA, 21 bp, Artificial Sequence
  Description of Artificial Sequence Synthetic oligonucleotide
ugagaauguacuaaccaggtt

SEQ ID NO: 139 - DNA, 21 bp, Artificial Sequence
  Description of Artificial Sequence Synthetic oligonucleotide
gagucuaaaaaugaccucatt

SEQ ID NO: 140 - DNA, 21 bp, Artificial Sequence
  Description of Artificial Sequence Synthetic oligonucleotide
ugaggucauuuuuagacuctt

SEQ ID NO: 141 - DNA, 21 bp, Artificial Sequence
  Description of Artificial Sequence Synthetic oligonucleotide
gaccucauauaguagugagtt

SEQ ID NO: 142 - DNA, 21 bp, Artificial Sequence
  Description of Artificial Sequence Synthetic oligonucleotide
cucacuacuauaugagguctt

SEQ ID NO: 143 - DNA, 21 bp, Artificial Sequence
  Description of Artificial Sequence Synthetic oligonucleotide
auaguagugaggaacauaatt

SEQ ID NO: 144 - DNA, 21 bp, Artificial Sequence
  Description of Artificial Sequence Synthetic oligonucleotide
uuauguuccucacuacuautt

SEQ ID NO: 145 - DNA, 21 bp, Artificial Sequence
  Description of Artificial Sequence Synthetic oligonucleotide
gggauuguacuauaccagutt

SEQ ID NO: 146 - DNA, 21 bp, Artificial Sequence
  Description of Artificial Sequence Synthetic oligonucleotide
acugguauaguacaauccctt

SEQ ID NO: 147 - DNA, 21 bp, Artificial Sequence
  Description of Artificial Sequence Synthetic oligonucleotide
ggauuguacuauaccaguatt

SEQ ID NO: 148 - DNA, 21 bp, Artificial Sequence
  Description of Artificial Sequence Synthetic oligonucleotide
uacugguauaguacaaucctt

SEQ ID NO: 149 - DNA, 21 bp, Artificial Sequence
  Description of Artificial Sequence Synthetic oligonucleotide
ggaaaugaguagaauugcutt

SEQ ID NO: 150 - DNA, 21 bp, Artificial Sequence
  Description of Artificial Sequence Synthetic oligonucleotide
agcaauucuacucauuucctt

SEQ ID NO: 151 - DNA, 21 bp, Artificial Sequence
  Description of Artificial Sequence Synthetic oligonucleotide
ggaccagugugcuucgucutt

SEQ ID NO: 152 - DNA, 21 bp, Artificial Sequence
  Description of Artificial Sequence Synthetic oligonucleotide
agacgaagcacacuggucctt

SEQ ID NO: 153 - DNA, 21 bp, Artificial Sequence
  Description of Artificial Sequence Synthetic oligonucleotide
ggacccuucauuagaucgctt

SEQ ID NO: 154 - DNA, 21 bp, Artificial Sequence
  Description of Artificial Sequence Synthetic oligonucleotide
gcgaucuaaugaagggucctt

SEQ ID NO: 155 - DNA, 21 bp, Artificial Sequence
  Description of Artificial Sequence Synthetic oligonucleotide
accucauauaguagugaggtt

SEQ ID NO: 156 - DNA, 21 bp, Artificial Sequence
  Description of Artificial Sequence Synthetic oligonucleotide
ccucacuacuauaugaggutt

SEQ ID NO: 157 - DNA, 21 bp, Artificial Sequence
  Description of Artificial Sequence Synthetic oligonucleotide
gaacguucgugguuccgugtt

SEQ ID NO: 158 - DNA, 21 bp, Artificial Sequence
  Description of Artificial Sequence Synthetic oligonucleotide
cacggaaccacgaacguuctt

SEQ ID NO: 159 - DNA, 21 bp, Artificial Sequence
  Description of Artificial Sequence Synthetic oligonucleotide
aauuccucgagauaggccgtt

SEQ ID NO: 160 - DNA, 21 bp, Artificial Sequence
  Description of Artificial Sequence Synthetic oligonucleotide
cggccuaucucgaggaauutt

SEQ ID NO: 161 - DNA, 21 bp, Artificial Sequence
  Description of Artificial Sequence Synthetic oligonucleotide
uuccucgagauaggccguutt

SEQ ID NO: 162 - DNA, 21 bp, Artificial Sequence
  Description of Artificial Sequence Synthetic oligonucleotide
aacggccuaucucgaggaatt

SEQ ID NO: 163 - DNA, 21 bp, Artificial Sequence
  Description of Artificial Sequence Synthetic oligonucleotide
gaacaauugcgagaacuautt

SEQ ID NO: 164 - DNA, 21 bp, Artificial Sequence
  Description of Artificial Sequence Synthetic oligonucleotide
auaguucucgcaauuguuctt

SEQ ID NO: 165 - DNA, 21 bp, Artificial Sequence
  Description of Artificial Sequence Synthetic oligonucleotide
gagaagaacguucgugguutt

SEQ ID NO: 166 - DNA, 21 bp, Artificial Sequence
  Description of Artificial Sequence Synthetic oligonucleotide
aaccacgaacguucuucuctt

SEQ ID NO: 167 - DNA, 21 bp, Artificial Sequence
  Description of Artificial Sequence Synthetic oligonucleotide
aagaacguucgugguuccgtt

SEQ ID NO: 168 - DNA, 21 bp, Artificial Sequence
  Description of Artificial Sequence Synthetic oligonucleotide
cggaaccacgaacguucuutt

SEQ ID NO: 169 - DNA, 21 bp, Artificial Sequence
  Description of Artificial Sequence Synthetic oligonucleotide
uugcucgacgauguuccautt

SEQ ID NO: 170 - DNA, 21 bp, Artificial Sequence
  Description of Artificial Sequence Synthetic oligonucleotide
auggaacaucgucgagcaatt

SEQ ID NO: 171 - DNA, 21 bp, Artificial Sequence
  Description of Artificial Sequence Synthetic oligonucleotide
aaacauuaucgcaacucagtt

SEQ ID NO: 172 - DNA, 21 bp, Artificial Sequence
  Description of Artificial Sequence Synthetic oligonucleotide
cugaguugcgauaauguuutt

SEQ ID NO: 173 - DNA, 21 bp, Artificial Sequence
  Description of Artificial Sequence Synthetic oligonucleotide
aacuguaauguuacgucautt

SEQ ID NO: 174 - DNA, 21 bp, Artificial Sequence
  Description of Artificial Sequence Synthetic oligonucleotide
augacguaacauuacaguutt

SEQ ID NO: 175 - DNA, 21 bp, Artificial Sequence
  Description of Artificial Sequence Synthetic oligonucleotide
gacccuucauuagaucgcctt

SEQ ID NO: 176 - DNA, 21 bp, Artificial Sequence
  Description of Artificial Sequence Synthetic oligonucleotide
ggcgaucuaaugaaggguctt

SEQ ID NO: 177 - DNA, 21 bp, Artificial Sequence
  Description of Artificial Sequence Synthetic oligonucleotide
guccacggcgagcggucuutt

SEQ ID NO: 178 - DNA, 21 bp, Artificial Sequence
  Description of Artificial Sequence Synthetic oligonucleotide
aagaccgcucgccguggactt

SEQ ID NO: 179 - DNA, 21 bp, Artificial Sequence
  Description of Artificial Sequence Synthetic oligonucleotide
cgagauaggccguuuguautt

SEQ ID NO: 180 - DNA, 21 bp, Artificial Sequence
  Description of Artificial Sequence Synthetic oligonucleotide
auacaaacggccuaucucgtt

SEQ ID NO: 181 - DNA, 21 bp, Artificial Sequence
  Description of Artificial Sequence Synthetic oligonucleotide
cuuacagcauugcggauuatt

SEQ ID NO: 182 - DNA, 21 bp, Artificial Sequence
  Description of Artificial Sequence Synthetic oligonucleotide
uaauccgcaaugcuguaagtt

SEQ ID NO: 183 - DNA, 21 bp, Artificial Sequence
  Description of Artificial Sequence Synthetic oligonucleotide
gcuuacagcauugcggauutt

SEQ ID NO: 184 - DNA, 21 bp, Artificial Sequence
  Description of Artificial Sequence Synthetic oligonucleotide
aauccgcaaugcuguaagctt

SEQ ID NO: 185 - DNA, 21 bp, Artificial Sequence
  Description of Artificial Sequence Synthetic oligonucleotide
uccucgagauaggccguuutt

SEQ ID NO: 186 - DNA, 21 bp, Artificial Sequence
  Description of Artificial Sequence Synthetic oligonucleotide
aaacggccuaucucgaggatt

SEQ ID NO: 187 - DNA, 21 bp, Artificial Sequence
  Description of Artificial Sequence Synthetic oligonucleotide
cucgagauaggccguuugutt

SEQ ID NO: 188 - DNA, 21 bp, Artificial Sequence
  Description of Artificial Sequence Synthetic oligonucleotide
acaaacggccuaucucgagtt

SEQ ID NO: 189 - DNA, 21 bp, Artificial Sequence
  Description of Artificial Sequence Synthetic oligonucleotide
acccuucauuagaucgccctt

SEQ ID NO: 190 - DNA, 21 bp, Artificial Sequence
  Description of Artificial Sequence Synthetic oligonucleotide
gggcgaucuaaugaagggutt

SEQ ID NO: 191 - DNA, 21 bp, Artificial Sequence
  Description of Artificial Sequence Synthetic oligonucleotide
uucagaggguacuacguugtt

SEQ ID NO: 192 - DNA, 21 bp, Artificial Sequence
  Description of Artificial Sequence Synthetic oligonucleotide
caacguaguacccucugaatt

SEQ ID NO: 193 - DNA, 21 bp, Artificial Sequence
  Description of Artificial Sequence Synthetic oligonucleotide
ucagaggguacuacguugatt

SEQ ID NO: 194 - DNA, 21 bp, Artificial Sequence
  Description of Artificial Sequence Synthetic oligonucleotide
ucaacguaguacccucugatt

SEQ ID NO: 195 - DNA, 21 bp, Artificial Sequence
  Description of Artificial Sequence Synthetic oligonucleotide
cagaggguacuacguugaatt

SEQ ID NO: 196 - DNA, 21 bp, Artificial Sequence
  Description of Artificial Sequence Synthetic oligonucleotide
uucaacguaguacccucugtt

SEQ ID NO: 197 - DNA, 21 bp, Artificial Sequence
  Description of Artificial Sequence Synthetic oligonucleotide
agaagaacguucgugguuctt

SEQ ID NO: 198 - DNA, 21 bp, Artificial Sequence
  Description of Artificial Sequence Synthetic oligonucleotide
gaaccacgaacguucuucutt

SEQ ID NO: 199 - DNA, 21 bp, Artificial Sequence
  Description of Artificial Sequence Synthetic oligonucleotide
acguucgugguuccgugagtt

SEQ ID NO: 200 - DNA, 21 bp, Artificial Sequence
  Description of Artificial Sequence Synthetic oligonucleotide
cucacggaaccacgaacgutt

SEQ ID NO: 201 - DNA, 21 bp, Artificial Sequence
  Description of Artificial Sequence Synthetic oligonucleotide
aaacuguaauguuacgucatt

SEQ ID NO: 202 - DNA, 21 bp, Artificial Sequence
  Description of Artificial Sequence Synthetic oligonucleotide
ugacguaacauuacaguuutt

SEQ ID NO: 203 - DNA, 21 bp, Artificial Sequence
  Description of Artificial Sequence Synthetic oligonucleotide
cuguauugcagacuuaggatt

SEQ ID NO: 204 - DNA, 21 bp, Artificial Sequence
  Description of Artificial Sequence Synthetic oligonucleotide
uccuaagucugcaauacagtt

SEQ ID NO: 205 - DNA, 21 bp, Artificial Sequence
  Description of Artificial Sequence Synthetic oligonucleotide
uuacuccugguuaguacautt

SEQ ID NO: 206 - DNA, 21 bp, Artificial Sequence
  Description of Artificial Sequence Synthetic oligonucleotide
auguacuaaccaggaguaatt

SEQ ID NO: 207 - DNA, 21 bp, Artificial Sequence
  Description of Artificial Sequence Synthetic oligonucleotide
ugaccucauauaguagugatt

SEQ ID NO: 208 - DNA, 21 bp, Artificial Sequence
  Description of Artificial Sequence Synthetic oligonucleotide
ucacuacuauaugaggucatt

SEQ ID NO: 209 - DNA, 21 bp, Artificial Sequence
  Description of Artificial Sequence Synthetic oligonucleotide
gguacuacguugaaagacutt

SEQ ID NO: 210 - DNA, 21 bp, Artificial Sequence
  Description of Artificial Sequence Synthetic oligonucleotide
agucuuucaacguaguacctt

SEQ ID NO: 211 - DNA, 21 bp, Artificial Sequence
  Description of Artificial Sequence Synthetic oligonucleotide
cauuaucgcaacucagucatt

SEQ ID NO: 212 - DNA, 21 bp, Artificial Sequence
  Description of Artificial Sequence Synthetic oligonucleotide
ugacugaguugcgauaaugtt

SEQ ID NO: 213 - DNA, 21 bp, Artificial Sequence
  Description of Artificial Sequence Synthetic oligonucleotide
gcgagaacuauuguguuactt

SEQ ID NO: 214 - DNA, 21 bp, Artificial Sequence
  Description of Artificial Sequence Synthetic oligonucleotide
guaacacaauaguucucgctt

SEQ ID NO: 215 - DNA, 21 bp, Artificial Sequence
  Description of Artificial Sequence Synthetic oligonucleotide
cgacggcguuacaguguuutt

SEQ ID NO: 216 - DNA, 21 bp, Artificial Sequence
  Description of Artificial Sequence Synthetic oligonucleotide
aaacacuguaacgccgucgtt

SEQ ID NO: 217 - DNA, 21 bp, Artificial Sequence
  Description of Artificial Sequence Synthetic oligonucleotide
agaacguucgugguuccgutt

SEQ ID NO: 218 - DNA, 21 bp, Artificial Sequence
  Description of Artificial Sequence Synthetic oligonucleotide
acggaaccacgaacguucutt

SEQ ID NO: 219 - DNA, 21 bp, Artificial Sequence
  Description of Artificial Sequence Synthetic oligonucleotide
ccucgagauaggccguuugtt

SEQ ID NO: 220 - DNA, 21 bp, Artificial Sequence
  Description of Artificial Sequence Synthetic oligonucleotide
caaacggccuaucucgaggtt

SEQ ID NO: 221 - DNA, 21 bp, Artificial Sequence
  Description of Artificial Sequence Synthetic oligonucleotide
gaucgcccuuuuauuucagtt

SEQ ID NO: 222 - DNA, 21 bp, Artificial Sequence
  Description of Artificial Sequence Synthetic oligonucleotide
cugaaauaaaagggcgauctt

SEQ ID NO: 223 - DNA, 21 bp, Artificial Sequence
  Description of Artificial Sequence Synthetic oligonucleotide
ucgcaacucagucaacaggtt

SEQ ID NO: 224 - DNA, 21 bp, Artificial Sequence
  Description of Artificial Sequence Synthetic oligonucleotide
ccuguugacugaguugcgatt

SEQ ID NO: 225 - DNA, 21 bp, Artificial Sequence
  Description of Artificial Sequence Synthetic oligonucleotide
acuaugaacgcuucuuucctt

SEQ ID NO: 226 - DNA, 21 bp, Artificial Sequence
  Description of Artificial Sequence Synthetic oligonucleotide
ggaaagaagcguucauagutt

SEQ ID NO: 227 - DNA, 21 bp, Artificial Sequence
  Description of Artificial Sequence Synthetic oligonucleotide
guacuauaccaguaagugctt

SEQ ID NO: 228 - DNA, 21 bp, Artificial Sequence
  Description of Artificial Sequence Synthetic oligonucleotide
gcacuuacugguauaguactt

SEQ ID NO: 229 - DNA, 21 bp, Artificial Sequence
  Description of Artificial Sequence Synthetic oligonucleotide
aauugacuuaauuccucgatt

SEQ ID NO: 230 - DNA, 21 bp, Artificial Sequence
  Description of Artificial Sequence Synthetic oligonucleotide
ucgaggaauuaagucaauutt

SEQ ID NO: 231 - DNA, 21 bp, Artificial Sequence
  Description of Artificial Sequence Synthetic oligonucleotide
uugacuuaauuccucgagatt

SEQ ID NO: 232 - DNA, 21 bp, Artificial Sequence
  Description of Artificial Sequence Synthetic oligonucleotide
ucucgaggaauuaagucaatt

SEQ ID NO: 233 - DNA, 21 bp, Artificial Sequence
  Description of Artificial Sequence Synthetic oligonucleotide
cugggucugugacuacaactt

SEQ ID NO: 234 - DNA, 21 bp, Artificial Sequence
  Description of Artificial Sequence Synthetic oligonucleotide
guuguagucacagacccagtt

SEQ ID NO: 235 - DNA, 21 bp, Artificial Sequence
  Description of Artificial Sequence Synthetic oligonucleotide
cugcaaucaggaccauugctt

SEQ ID NO: 236 - DNA, 21 bp, Artificial Sequence
  Description of Artificial Sequence Synthetic oligonucleotide
gcaaugguccugauugcagtt

SEQ ID NO: 237 - DNA, 21 bp, Artificial Sequence
  Description of Artificial Sequence Synthetic oligonucleotide
gaccagugugcuucgucugtt

SEQ ID NO: 238 - DNA, 21 bp, Artificial Sequence
  Description of Artificial Sequence Synthetic oligonucleotide
cagacgaagcacacugguctt

SEQ ID NO: 239 - DNA, 21 bp, Artificial Sequence
  Description of Artificial Sequence Synthetic oligonucleotide
cuauaucugccacaaccgctt

SEQ ID NO: 240 - DNA, 21 bp, Artificial Sequence
  Description of Artificial Sequence Synthetic oligonucleotide
gcgguuguggcagauauagtt

SEQ ID NO: 241 - DNA, 21 bp, Artificial Sequence
  Description of Artificial Sequence Synthetic oligonucleotide
aaccgcacugucauucacctt

SEQ ID NO: 242 - DNA, 21 bp, Artificial Sequence
  Description of Artificial Sequence Synthetic oligonucleotide
ggugaaugacagugcgguutt

SEQ ID NO: 243 - DNA, 21 bp, Artificial Sequence
  Description of Artificial Sequence Synthetic oligonucleotide
cauucaccaucgagugccatt

SEQ ID NO: 244 - DNA, 21 bp, Artificial Sequence
  Description of Artificial Sequence Synthetic oligonucleotide
uggcacucgauggugaaugtt

SEQ ID NO: 245 - DNA, 21 bp, Artificial Sequence
  Description of Artificial Sequence Synthetic oligonucleotide
agaggguacuacguugaaatt

SEQ ID NO: 246 - DNA, 21 bp, Artificial Sequence
  Description of Artificial Sequence Synthetic oligonucleotide
uuucaacguaguacccucutt

SEQ ID NO: 247 - DNA, 21 bp, Artificial Sequence
  Description of Artificial Sequence Synthetic oligonucleotide
auuuaugauaugacaacgutt

SEQ ID NO: 248 - DNA, 21 bp, Artificial Sequence
  Description of Artificial Sequence Synthetic oligonucleotide
acguugucauaucauaaautt

SEQ ID NO: 249 - DNA, 21 bp, Artificial Sequence
  Description of Artificial Sequence Synthetic oligonucleotide
cauuggcaaaggucgauuutt

SEQ ID NO: 250 - DNA, 21 bp, Artificial Sequence
  Description of Artificial Sequence Synthetic oligonucleotide
aaaucgaccuuugccaaugtt

SEQ ID NO: 251 - DNA, 21 bp, Artificial Sequence
  Description of Artificial Sequence Synthetic oligonucleotide
ugguuggugucagauuauctt

SEQ ID NO: 252 - DNA, 21 bp, Artificial Sequence
  Description of Artificial Sequence Synthetic oligonucleotide
gauaaucugacaccaaccatt

SEQ ID NO: 253 - DNA, 21 bp, Artificial Sequence
  Description of Artificial Sequence Synthetic oligonucleotide
gguuggugucagauuaucatt

SEQ ID NO: 254 - DNA, 21 bp, Artificial Sequence
  Description of Artificial Sequence Synthetic oligonucleotide
ugauaaucugacaccaacctt

SEQ ID NO: 255 - DNA, 21 bp, Artificial Sequence
  Description of Artificial Sequence Synthetic oligonucleotide
uuaucaugagcauggaucctt

SEQ ID NO: 256 - DNA, 21 bp, Artificial Sequence
  Description of Artificial Sequence Synthetic oligonucleotide
ggauccaugcucaugauaatt

SEQ ID NO: 257 - DNA, 21 bp, Artificial Sequence
  Description of Artificial Sequence Synthetic oligonucleotide
agcauggaucccuuuuugatt

SEQ ID NO: 258 - DNA, 21 bp, Artificial Sequence
  Description of Artificial Sequence Synthetic oligonucleotide
ucaaaaagggauccaugcutt

SEQ ID NO: 259 - DNA, 21 bp, Artificial Sequence
  Description of Artificial Sequence Synthetic oligonucleotide
caaugggcuuaguauucugtt

SEQ ID NO: 260 - DNA, 21 bp, Artificial Sequence
  Description of Artificial Sequence Synthetic oligonucleotide
cagaauacuaagcccauugtt

SEQ ID NO: 261 - DNA, 21 bp, Artificial Sequence
  Description of Artificial Sequence Synthetic oligonucleotide
cugggaaauugcucgacgatt

SEQ ID NO: 262 - DNA, 21 bp, Artificial Sequence
  Description of Artificial Sequence Synthetic oligonucleotide
ucgucgagcaauuucccagtt

SEQ ID NO: 263 - DNA, 21 bp, Artificial Sequence
  Description of Artificial Sequence Synthetic oligonucleotide
cucgacgauguuccauuggtt

SEQ ID NO: 264 - DNA, 21 bp, Artificial Sequence
  Description of Artificial Sequence Synthetic oligonucleotide
ccaauggaacaucgucgagtt

SEQ ID NO: 265 - DNA, 21 bp, Artificial Sequence
  Description of Artificial Sequence Synthetic oligonucleotide
ucgacgauguuccauuggutt

SEQ ID NO: 266 - DNA, 21 bp, Artificial Sequence
  Description of Artificial Sequence Synthetic oligonucleotide
accaauggaacaucgucgatt

SEQ ID NO: 267 - DNA, 21 bp, Artificial Sequence
  Description of Artificial Sequence Synthetic oligonucleotide
cgacgauguuccauuggugtt

SEQ ID NO: 268 - DNA, 21 bp, Artificial Sequence
  Description of Artificial Sequence Synthetic oligonucleotide
caccaauggaacaucgucgtt

SEQ ID NO: 269 - DNA, 21 bp, Artificial Sequence
  Description of Artificial Sequence Synthetic oligonucleotide
gacgauguuccauugguggtt

SEQ ID NO: 270 - DNA, 21 bp, Artificial Sequence
  Description of Artificial Sequence Synthetic oligonucleotide
ccaccaauggaacaucguctt

SEQ ID NO: 271 - DNA, 21 bp, Artificial Sequence
  Description of Artificial Sequence Synthetic oligonucleotide
gaaaacauuaucgcaacuctt

SEQ ID NO: 272 - DNA, 21 bp, Artificial Sequence
  Description of Artificial Sequence Synthetic oligonucleotide
gaguugcgauaauguuuuctt

SEQ ID NO: 273 - DNA, 21 bp, Artificial Sequence
  Description of Artificial Sequence Synthetic oligonucleotide
aaaacauuaucgcaacucatt

SEQ ID NO: 274 - DNA, 21 bp, Artificial Sequence
  Description of Artificial Sequence Synthetic oligonucleotide
ugaguugcgauaauguuuutt

SEQ ID NO: 275 - DNA, 21 bp, Artificial Sequence
  Description of Artificial Sequence Synthetic oligonucleotide
uaucgcaacucagucaacatt

SEQ ID NO: 276 - DNA, 21 bp, Artificial Sequence
  Description of Artificial Sequence Synthetic oligonucleotide
uguugacugaguugcgauatt

SEQ ID NO: 277 - DNA, 21 bp, Artificial Sequence
  Description of Artificial Sequence Synthetic oligonucleotide
cuacagcuuugccugaacutt

SEQ ID NO: 278 - DNA, 21 bp, Artificial Sequence
  Description of Artificial Sequence Synthetic oligonucleotide
aguucaggcaaagcuguagtt

SEQ ID NO: 279 - DNA, 21 bp, Artificial Sequence
  Description of Artificial Sequence Synthetic oligonucleotide
uucugugcacuaugaacgctt

SEQ ID NO: 280 - DNA, 21 bp, Artificial Sequence
  Description of Artificial Sequence Synthetic oligonucleotide
gcguucauagugcacagaatt

SEQ ID NO: 281 - DNA, 21 bp, Artificial Sequence
  Description of Artificial Sequence Synthetic oligonucleotide
ugcacuaugaacgcuucuutt

SEQ ID NO: 282 - DNA, 21 bp, Artificial Sequence
  Description of Artificial Sequence Synthetic oligonucleotide
aagaagcguucauagugcatt

SEQ ID NO: 283 - DNA, 21 bp, Artificial Sequence
  Description of Artificial Sequence Synthetic oligonucleotide
ugauuuacuccugguuagutt

SEQ ID NO: 284 - DNA, 21 bp, Artificial Sequence
  Description of Artificial Sequence Synthetic oligonucleotide
acuaaccaggaguaaaucatt

SEQ ID NO: 285 - DNA, 21 bp, Artificial Sequence
  Description of Artificial Sequence Synthetic oligonucleotide
uuuacuccugguuaguacatt

SEQ ID NO: 286 - DNA, 21 bp, Artificial Sequence
  Description of Artificial Sequence Synthetic oligonucleotide
uguacuaaccaggaguaaatt

SEQ ID NO: 287 - DNA, 21 bp, Artificial Sequence
  Description of Artificial Sequence Synthetic oligonucleotide
ccugguuaguacauucucatt

SEQ ID NO: 288 - DNA, 21 bp, Artificial Sequence
  Description of Artificial Sequence Synthetic oligonucleotide
ugagaauguacuaaccaggtt

SEQ ID NO: 289 - DNA, 21 bp, Artificial Sequence
  Description of Artificial Sequence Synthetic oligonucleotide
gagucuaaaaaugaccucatt

SEQ ID NO: 290 - DNA, 21 bp, Artificial Sequence
  Description of Artificial Sequence Synthetic oligonucleotide
ugaggucauuuuuagacuctt

SEQ ID NO: 291 - DNA, 21 bp, Artificial Sequence
  Description of Artificial Sequence Synthetic oligonucleotide
gaccucauauaguagugagtt

SEQ ID NO: 292 - DNA, 21 bp, Artificial Sequence
  Description of Artificial Sequence Synthetic oligonucleotide
cucacuacuauaugagguctt

SEQ ID NO: 293 - DNA, 21 bp, Artificial Sequence
  Description of Artificial Sequence Synthetic oligonucleotide
auaguagugaggaacauaatt

SEQ ID NO: 294 - DNA, 21 bp, Artificial Sequence
  Description of Artificial Sequence Synthetic oligonucleotide
uuauguuccucacuacuautt

SEQ ID NO: 295 - DNA, 21 bp, Artificial Sequence
  Description of Artificial Sequence Synthetic oligonucleotide
gggauuguacuauaccagutt

SEQ ID NO: 296 - DNA, 21 bp, Artificial Sequence
  Description of Artificial Sequence Synthetic oligonucleotide
acugguauaguacaauccctt

SEQ ID NO: 297 - DNA, 21 bp, Artificial Sequence
  Description of Artificial Sequence Synthetic oligonucleotide
ggauuguacuauaccaguatt

SEQ ID NO: 298 - DNA, 21 bp, Artificial Sequence
  Description of Artificial Sequence Synthetic oligonucleotide
uacugguauaguacaaucctt

SEQ ID NO: 299 - DNA, 21 bp, Artificial Sequence
  Description of Artificial Sequence Synthetic oligonucleotide
ggaaaugaguagaauugcutt

SEQ ID NO: 300 - DNA, 21 bp, Artificial Sequence
  Description of Artificial Sequence Synthetic oligonucleotide
agcaauucuacucauuucctt

SEQ ID NO: 301 - DNA, 43 bp, Artificial Sequence
  Description of Artificial Sequence Synthetic oligonucleotide
cgtcgagcaatttcccagaatatttttctcttggaaagaaagt

SEQ ID NO: 302 - DNA, 46 bp, Artificial Sequence
  Description of Artificial Sequence Synthetic oligonucleotide
tgggatatttggccttaacttctgttttttctcttggaaagaaagt

SEQ ID NO: 303 - DNA, 43 bp, Artificial Sequence
  Description of Artificial Sequence Synthetic oligonucleotide
caatgctgtaagcctagctgcttttttctcttggaaagaaagt

SEQ ID NO: 304 - DNA, 45 bp, Artificial Sequence
  Description of Artificial Sequence Synthetic oligonucleotide
ttgcgataatgttttcttaatccgtttttctcttggaaagaaagt

SEQ ID NO: 305 - DNA, 43 bp, Artificial Sequence
  Description of Artificial Sequence Synthetic oligonucleotide
tgatgccttcctgttgactgagtttttctcttggaaagaaagt

SEQ ID NO: 306 - DNA, 48 bp, Artificial Sequence
  Description of Artificial Sequence Synthetic oligonucleotide
ctaagcccattgcatagatgtcagtttttaggcataggacccgtgtct

SEQ ID NO: 307 - DNA, 50 bp, Artificial Sequence
  Description of Artificial Sequence Synthetic oligonucleotide
tcacaaacaacttttctcatttcttctttttaggcataggacccgtgtct

SEQ ID NO: 308 - DNA, 46 bp, Artificial Sequence
  Description of Artificial Sequence Synthetic oligonucleotide
cttcacagctctgccatctgtttttttaggcataggacccgtgtct

SEQ ID NO: 309 - DNA, 50 bp, Artificial Sequence
  Description of Artificial Sequence Synthetic oligonucleotide
cataattttagccattactctcaaggtttttaggcataggacccgtgtct

SEQ ID NO: 310 - DNA, 23 bp, Artificial Sequence
  Description of Artificial Sequence Synthetic oligonucleotide
catgaattccaccaatggaacat

SEQ ID NO: 311 - DNA, 26 bp, Artificial Sequence
  Description of Artificial Sequence Synthetic oligonucleotide
atcataataaggcagttggtaatctt

SEQ ID NO: 312 - DNA, 24 bp, Artificial Sequence
  Description of Artificial Sequence Synthetic oligonucleotide
aactgatgggtcagaaggtacaag

SEQ ID NO: 313 - DNA, 23 bp, Artificial Sequence
  Description of Artificial Sequence Synthetic oligonucleotide
ccattggcataccaacattctct

SEQ ID NO: 314 - DNA, 24 bp, Artificial Sequence
  Description of Artificial Sequence Synthetic oligonucleotide
aactgatgggtcagaaggtacaag

SEQ ID NO: 315 - DNA, 41 bp, Artificial Sequence
  Description of Artificial Sequence Synthetic oligonucleotide
gaatttgccatgggtggaattttttctcttggaaagaaagt

SEQ ID NO: 316 - DNA, 41 bp, Artificial Sequence
  Description of Artificial Sequence Synthetic oligonucleotide
ggagggatctcgctcctggatttttctcttggaaagaaagt

SEQ ID NO: 317 - DNA, 40 bp, Artificial Sequence
  Description of Artificial Sequence Synthetic oligonucleotide
ccccagccttctccatggttttttctcttggaaagaaagt

SEQ ID NO: 318 - DNA, 40 bp, Artificial Sequence
  Description of Artificial Sequence Synthetic oligonucleotide
gctcccccctgcaaatgagtttttctcttggaaagaaagt

SEQ ID NO: 319 - DNA, 42 bp, Artificial Sequence
  Description of Artificial Sequence Synthetic oligonucleotide
agccttgacggtgccatgtttttaggcataggacccgtgtct

SEQ ID NO: 320 - DNA, 45 bp, Artificial Sequence
  Description of Artificial Sequence Synthetic oligonucleotide
gatgacaagcttcccgttctctttttaggcataggacccgtgtct

SEQ ID NO: 321 - DNA, 46 bp, Artificial Sequence
  Description of Artificial Sequence Synthetic oligonucleotide
agatggtgatgggatttccatttttttaggcataggacccgtgtct

SEQ ID NO: 322 - DNA, 44 bp, Artificial Sequence
  Description of Artificial Sequence Synthetic oligonucleotide
gcatcgccccacttgatttttttttaggcataggacccgtgtct

SEQ ID NO: 323 - DNA, 43 bp, Artificial Sequence
  Description of Artificial Sequence Synthetic oligonucleotide
cacgacgtactcagcgccatttttaggcataggacccgtgtct

SEQ ID NO: 324 - DNA, 46 bp, Artificial Sequence
  Description of Artificial Sequence Synthetic oligonucleotide
ggcagagatgatgacccttttgtttttaggcataggacccgtgtct

SEQ ID NO: 325 - DNA, 21 bp, Artificial Sequence
  Description of Artificial Sequence Synthetic oligonucleotide
ggtgaagacgccagtggactc

SEQ ID NO: 326 - DNA, 6475 bp, Homo sapiens
ggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatctctgccttttaagatatgtacagaaaatgtccatataaatttccattgaagtcgaatgatactgagaagcctgtaaagaggagaaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaagtgccagtggcatattttaaaataaagtgtatacgttggaatgagtcatgccatatgtagttgctgtagatggcaactagaacctttgagttacaagagtctttagaagttttctaaccctgcctagtgcaagttacaatattatagcgtgttcggggagtgccctcctgtctgcaggtgtgtctctgtgcctgggggcttttctccacatgcttaggggtgtgggtcttccattggggcatgatggacctgtctacaggtgatctctgttgcctttgggtcagcacatttgttagtctcctgggggtgaaaacttggcttacaagagaactggaaaaatgatgagatgtggtccccaaacccttgattgactctggggaggggctttgtgaataggattgctctcacattaaagatagttacttcaatttgaaggctggatttagggatttttttttttccttataacaaagacatcaccaggatatgaagcttttgttgaaagttggaaaaaaagtgaaattaaagacattcccagacaaa

SEQ ID NO: 327 - RNA, 23 nt, Homo sapiens
cuggaccagugugcuucgucugc

SEQ ID NO: 328 - RNA, 23 nt, Homo sapiens
cucgacgauguuccauuggugga

SEQ ID NO: 329 - RNA, 23 nt, Homo sapiens
cugguuggugucagauuaucaug

SEQ ID NO: 330 - RNA, 23 nt, Homo sapiens
ggcuuacagcauugcggauuaag

SEQ ID NO: 331 - RNA, 23 nt, Homo sapiens
aauugacuuaauuccucgagaua

SEQ ID NO: 332 - RNA, 23 nt, Homo sapiens
agcauuggcaaaggucgauuugg

SEQ ID NO: 333 - RNA, 23 nt, Homo sapiens
caaacuguaauguuacgucauga

SEQ ID NO: 334 - RNA, 23 nt, Homo sapiens
gagaacaauugcgagaacuauug

SEQ ID NO: 335 - RNA, 23 nt, Homo sapiens
cucgagauaggccguuuguaugu

SEQ ID NO: 336 - RNA, 23 nt, Homo sapiens
aagaacguucgugguuccgugag

SEQ ID NO: 337 - RNA, 23 nt, Homo sapiens
uucugggaaauugcucgacgaug

SEQ ID NO: 338 - RNA, 23 nt, Homo sapiens
agaaaacauuaucgcaacucagu

SEQ ID NO: 339 - RNA, 23 nt, Homo sapiens
uuccucgagauaggccguuugua

SEQ ID NO: 340 - RNA, 23 nt, Homo sapiens
aacauuaucgcaacucagucaac

SEQ ID NO: 341 - RNA, 23 nt, Homo sapiens
uugcgagaacuauuguguuacaa

SEQ ID NO: 342 - RNA, 23 nt, Homo sapiens
ggcgacggcguuacaguguuucu

SEQ ID NO: 343 - RNA, 23 nt, Homo sapiens
uagagaagaacguucgugguucc

SEQ ID NO: 344 - RNA, 23 nt, Homo sapiens
ucagaggguacuacguugaaaga

SEQ ID NO: 345 - RNA, 23 nt, Homo sapiens
auggaaaugaguagaauugcuga

SEQ ID NO: 346 - RNA, 23 nt, Homo sapiens
uggaccagugugcuucgucugca

SEQ ID NO: 347 - RNA, 23 nt, Homo sapiens
aggguacuacguugaaagacuua

SEQ ID NO: 348 - RNA, 23 nt, Homo sapiens
ucaaacuguaauguuacgucaug

SEQ ID NO: 349 - RNA, 23 nt, Homo sapiens
ugcuguauugcagacuuaggacu

SEQ ID NO: 350 - RNA, 23 nt, Homo sapiens
auuccucgagauaggccguuugu

SEQ ID NO: 351 - RNA, 23 nt, Homo sapiens
augggauuguacuauaccaguaa

SEQ ID NO: 352 - RNA, 23 nt, Homo sapiens
ugcaaugggcuuaguauucuggg

SEQ ID NO: 353 - RNA, 23 nt, Homo sapiens
gaaauugacuuaauuccucgaga

SEQ ID NO: 354 - RNA, 23 nt, Homo sapiens
aacugggucugugacuacaacau

SEQ ID NO: 355 - RNA, 23 nt, Homo sapiens
cuccugguuaguacauucucaga

SEQ ID NO: 356 - RNA, 23 nt, Homo sapiens
ugugcacuaugaacgcuucuuuc

SEQ ID NO: 357 - RNA, 23 nt, Homo sapiens
ugagcauggaucccuuuuugauu

SEQ ID NO: 358 - RNA, 23 nt, Homo sapiens
gaaaacauuaucgcaacucaguc

SEQ ID NO: 359 - RNA, 23 nt, Homo sapiens
cuguccacggcgagcggucuugc

SEQ ID NO: 360 - RNA, 23 nt, Homo sapiens
agagaagaacguucgugguuccg

SEQ ID NO: 361 - RNA, 23 nt, Homo sapiens
auuaucgcaacucagucaacagg

SEQ ID NO: 362 - RNA, 23 nt, Homo sapiens
gaagaacguucgugguuccguga

SEQ ID NO: 363 - RNA, 23 nt, Homo sapiens
aggcuuacagcauugcggauuaa

SEQ ID NO: 364 - RNA, 23 nt, Homo sapiens
auauaguagugaggaacauaauu

SEQ ID NO: 365 - RNA, 23 nt, Homo sapiens
uccucgagauaggccguuuguau

SEQ ID NO: 366 - RNA, 23 nt, Homo sapiens
aaugaccucauauaguagugagg

SEQ ID NO: 367 - RNA, 23 nt, Homo sapiens
gucauucaccaucgagugccaaa

SEQ ID NO: 368 - RNA, 23 nt, Homo sapiens
gcucgacgauguuccauuggugg

SEQ ID NO: 369 - RNA, 23 nt, Homo sapiens
aauuccucgagauaggccguuug

SEQ ID NO: 370 - RNA, 23 nt, Homo sapiens
gauuuacuccugguuaguacauu

SEQ ID NO: 371 - RNA, 23 nt, Homo sapiens
uucuacagcuuugccugaacucu

SEQ ID NO: 372 - RNA, 23 nt, Homo sapiens
ugggauuguacuauaccaguaag

SEQ ID NO: 373 - RNA, 23 nt, Homo sapiens
ucgacgauguuccauugguggaa

SEQ ID NO: 374 - RNA, 23 nt, Homo sapiens
auuuacuccugguuaguacauuc

SEQ ID NO: 375 - RNA, 23 nt, Homo sapiens
gucuauaucugccacaaccgcac

SEQ ID NO: 376 - RNA, 23 nt, Homo sapiens
ucugguuggugucagauuaucau

SEQ ID NO: 377 - RNA, 23 nt, Homo sapiens
augaccucauauaguagugagga

SEQ ID NO: 378 - RNA, 23 nt, Homo sapiens
agugauuuacuccugguuaguac

SEQ ID NO: 379 - RNA, 23 nt, Homo sapiens
aauugcucgacgauguuccauug

SEQ ID NO: 380 - RNA, 23 nt, Homo sapiens
aggacccuucauuagaucgcccu

SEQ ID NO: 381 - RNA, 23 nt, Homo sapiens
ugcugcaaucaggaccauugcaa

SEQ ID NO: 382 - RNA, 23 nt, Homo sapiens
uuguacuauaccaguaagugcca

SEQ ID NO: 383 - RNA, 23 nt, Homo sapiens
uugagucuaaaaaugaccucaua

SEQ ID NO: 384 - RNA, 23 nt, Homo sapiens
ugcucgacgauguuccauuggug

SEQ ID NO: 385 - RNA, 23 nt, Homo sapiens
uuucagaggguacuacguugaaa

SEQ ID NO: 386 - RNA, 23 nt, Homo sapiens
ugaccucauauaguagugaggaa

SEQ ID NO: 387 - RNA, 23 nt, Homo sapiens
uaucgcaacucagucaacaggaa

SEQ ID NO: 388 - RNA, 23 nt, Homo sapiens
gcacuaugaacgcuucuuuccca

SEQ ID NO: 389 - RNA, 23 nt, Homo sapiens
aagaaaacauuaucgcaacucag

SEQ ID NO: 390 - RNA, 23 nt, Homo sapiens
uucagaggguacuacguugaaag

SEQ ID NO: 391 - RNA, 23 nt, Homo sapiens
uaauuuaugauaugacaacguca

SEQ ID NO: 392 - RNA, 23 nt, Homo sapiens
auuucagaggguacuacguugaa

SEQ ID NO: 393 - RNA, 23 nt, Homo sapiens
uagaucgcccuuuuauuucagag

SEQ ID NO: 394 - RNA, 23 nt, Homo sapiens
gaggacccuucauuagaucgccc

SEQ ID NO: 395 - RNA, 23 nt, Homo sapiens
cuuucugugcacuaugaacgcuu

SEQ ID NO: 396 - RNA, 23 nt, Homo sapiens
gaacguucgugguuccgugaggc

SEQ ID NO: 397 - RNA, 23 nt, Homo sapiens
gauuaucaugagcauggaucccu

SEQ ID NO: 398 - RNA, 23 nt, Homo sapiens
uuaauuccucgagauaggccguu

SEQ ID NO: 399 - RNA, 23 nt, Homo sapiens
ggacccuucauuagaucgcccuu

SEQ ID NO: 400 - RNA, 23 nt, Homo sapiens
acaaccgcacugucauucaccau

SEQ ID NO: 401 - RNA, 23 nt, Homo sapiens
agaagaacguucgugguuccgug

(19) United States
(12) Patent Application Publication
(10) Pub. No.: US 2011/0119781 A1
(43) Pub. Date: May 19, 2011

(54) Compositions and Methods for Inhibiting Expression of TGF-BETA Receptor Genes

(76) Inventors: Birgit Bramlage, Kulmbach (DE); Markus Hossbach, Kulmbach (DE); Pamela Tan, Kulmbach (DE); Hans-Peter Vamlocher, Bayreuth (DE)

(21) Appl. No.: 13/054,111

(22) PCT Filed: Jul. 8, 2009
(86) PCT No.: PCT/EP2009/058660

§ 371 Date: Jan. 14, 2011

(30) Foreign Application Priority Data
Jul. 15, 2008 (EP) 08012777.2

(51) Int. Cl. A01K 67/027; C07H 21/02; C12N 5/10

(52) U.S. Cl. 800/18; 536/23.1; 435/354

(57) ABSTRACT

The invention relates to a double-stranded ribonucleic acid (dsRNA) for inhibiting the expression of a TGF-beta receptor type I gene, comprising an antisense strand having a nucleotide sequence which is less than 30 nucleotides in length and which is substantially complementary to at least a part of a TGF-beta receptor type I gene. The invention also relates to a pharmaceutical composition comprising the dsRNA or nucleic acid molecules or vectors encoding the same together with a pharmaceutically acceptable carrier; methods for treating diseases caused by the expression of a TGF-beta receptor type I gene using said pharmaceutical composition; and methods for inhibiting the expression of a TGF-beta receptor type I gene in a cell.

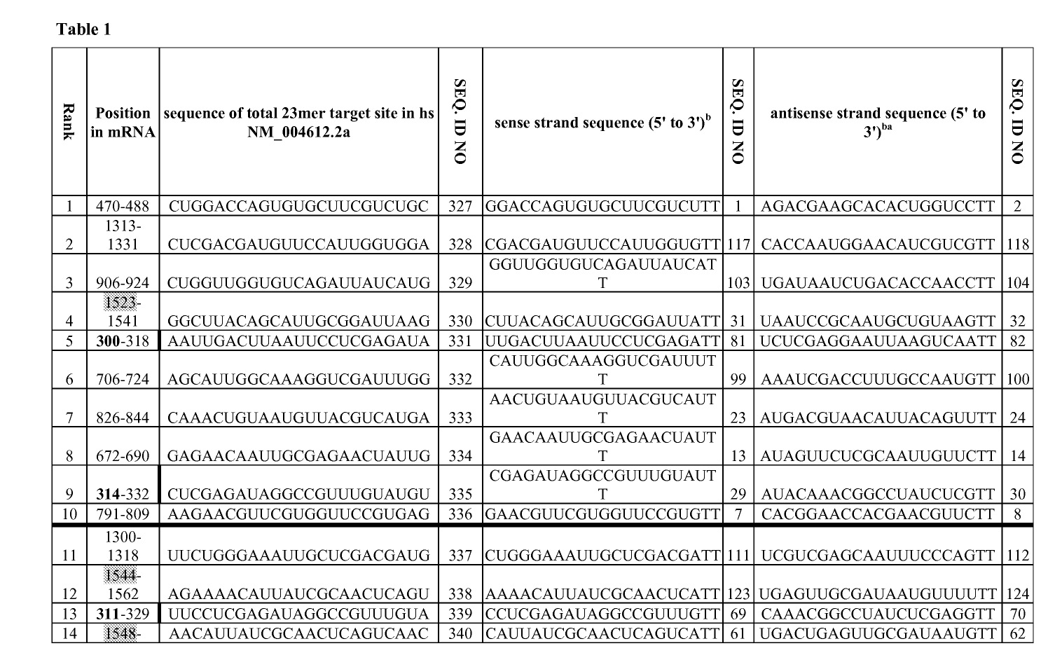

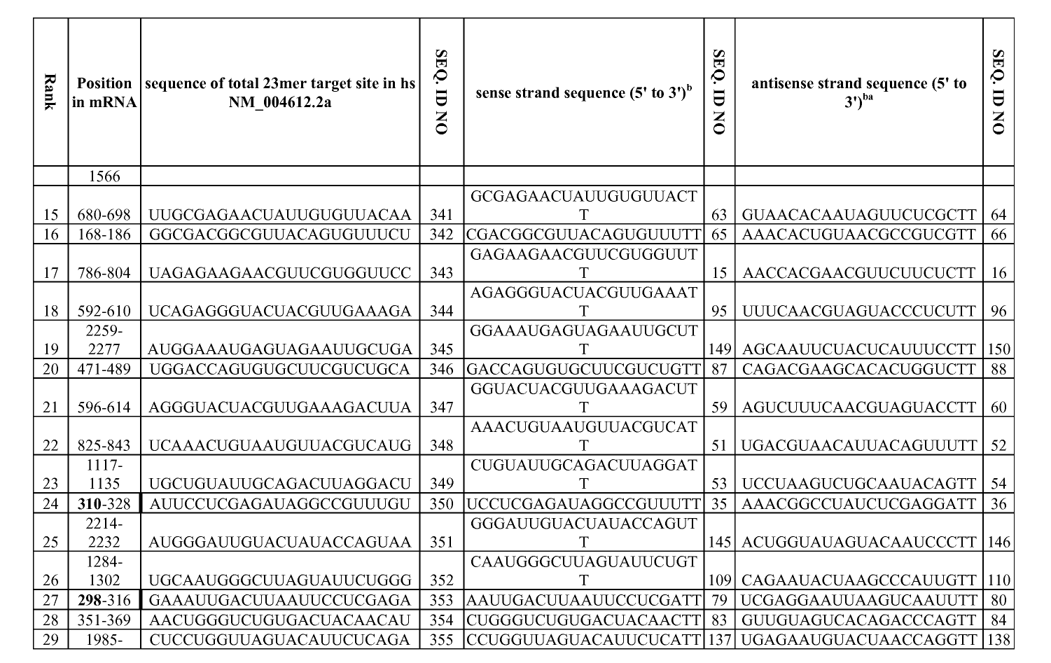

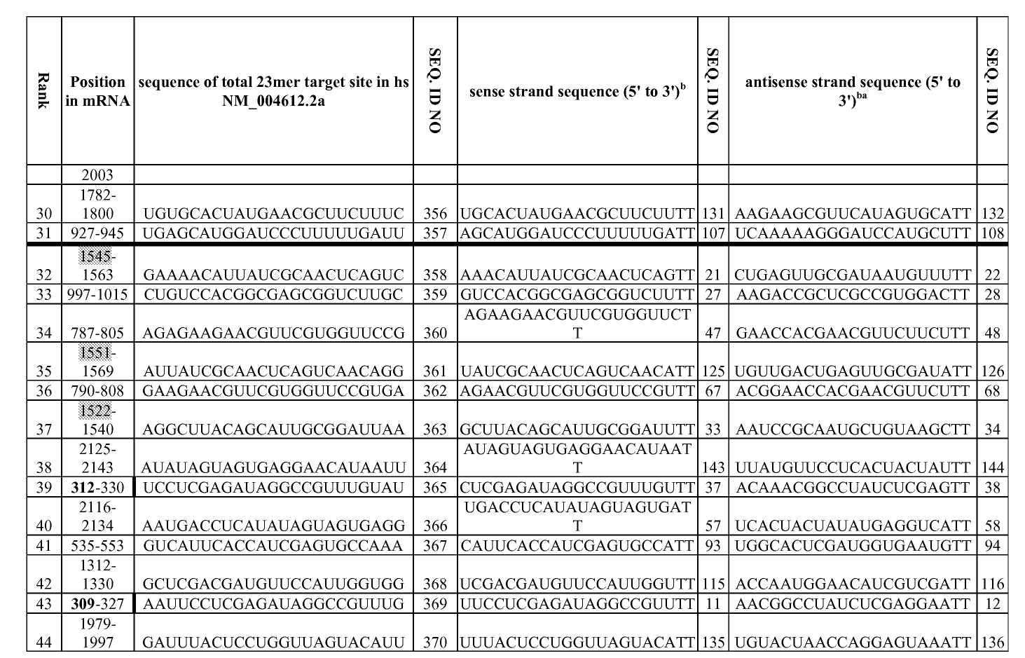

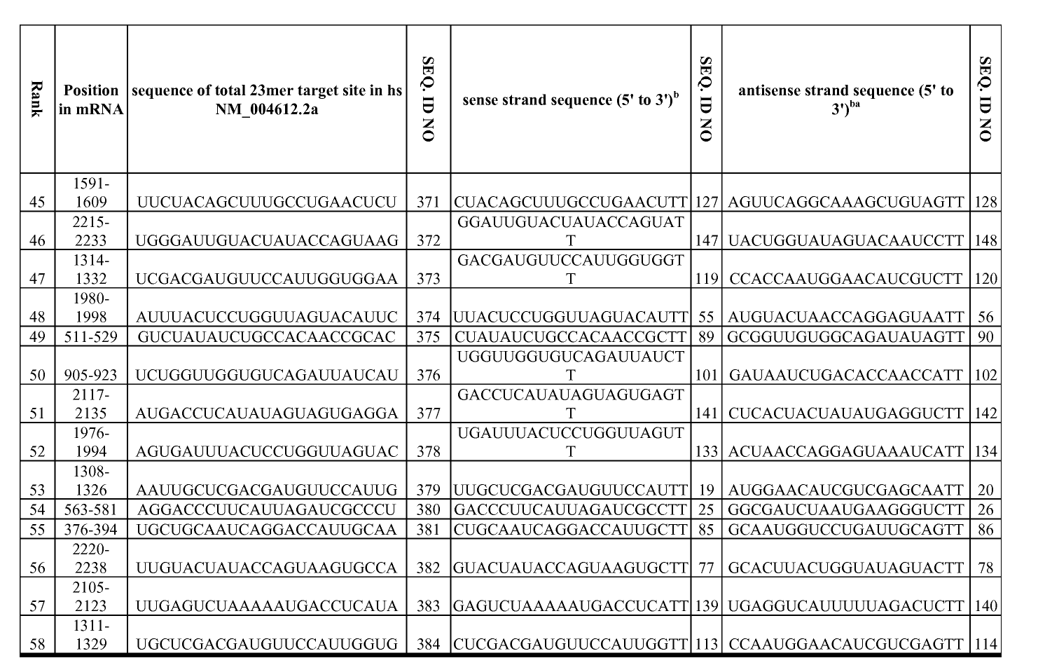

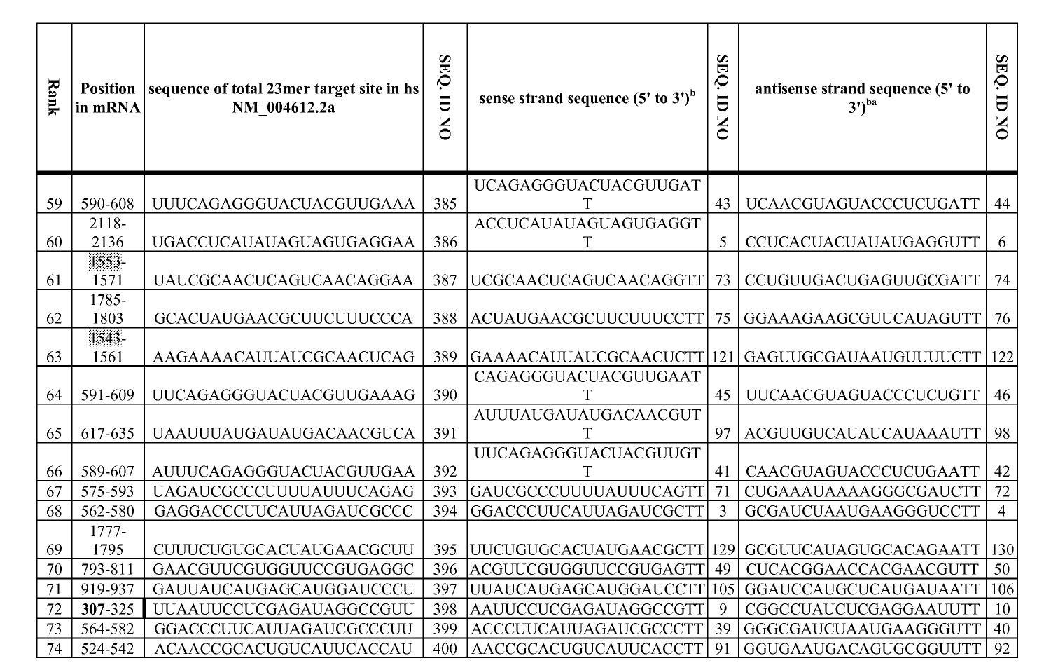

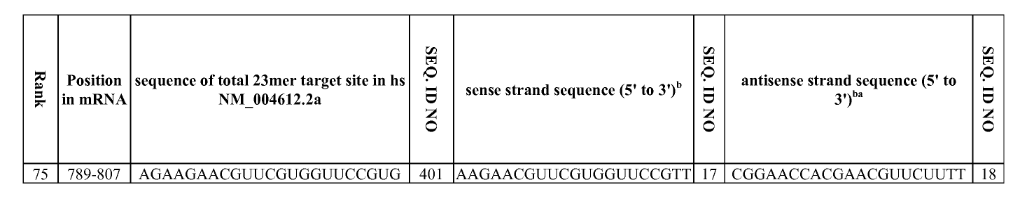

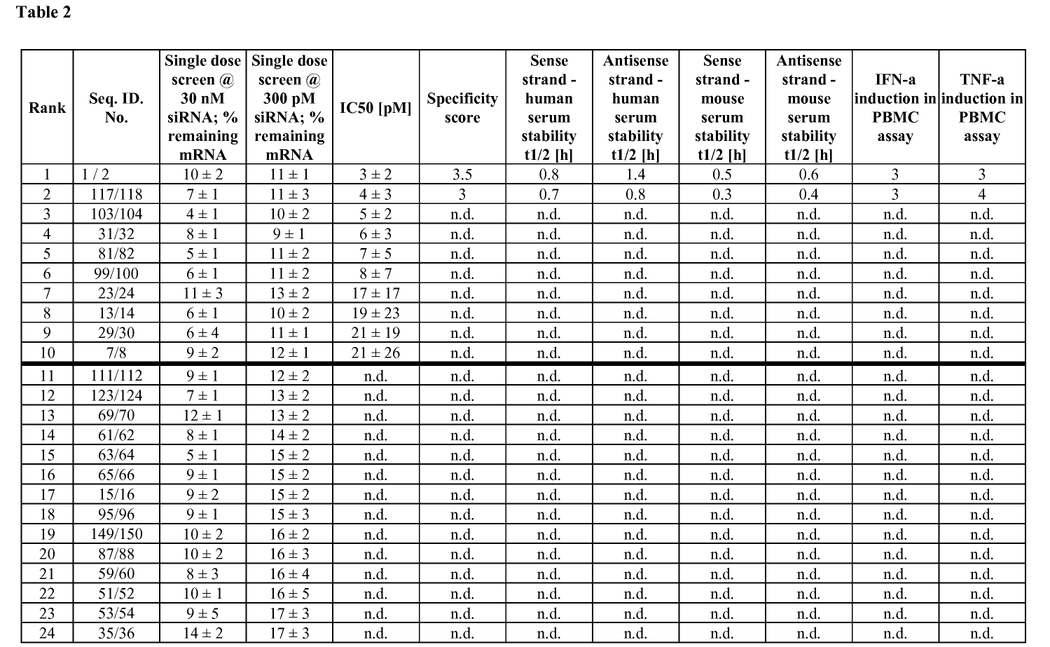

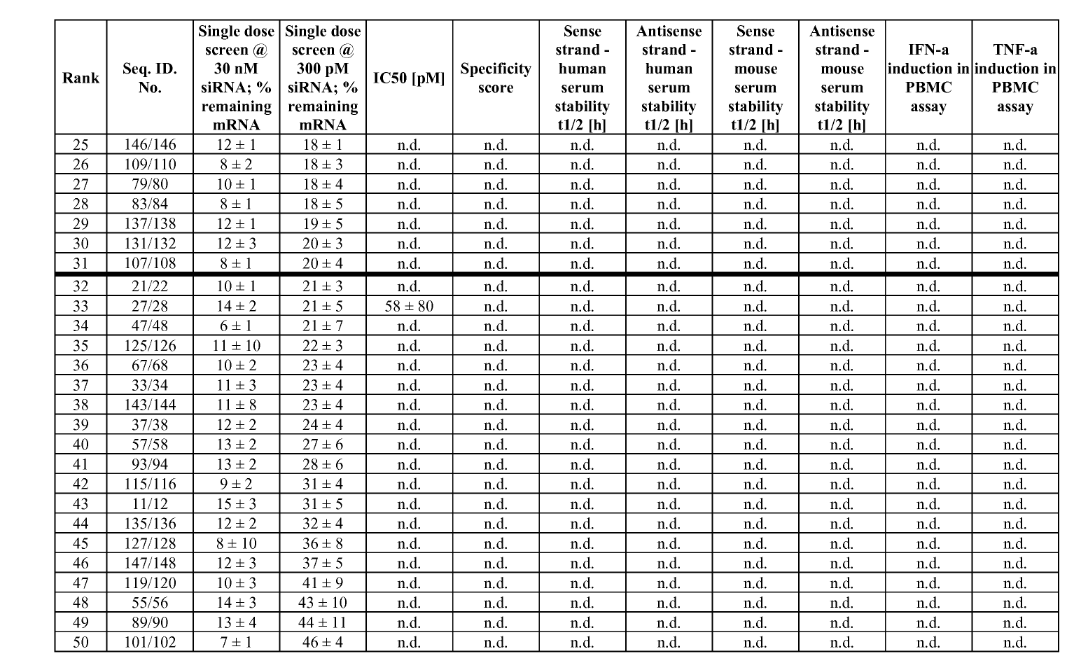

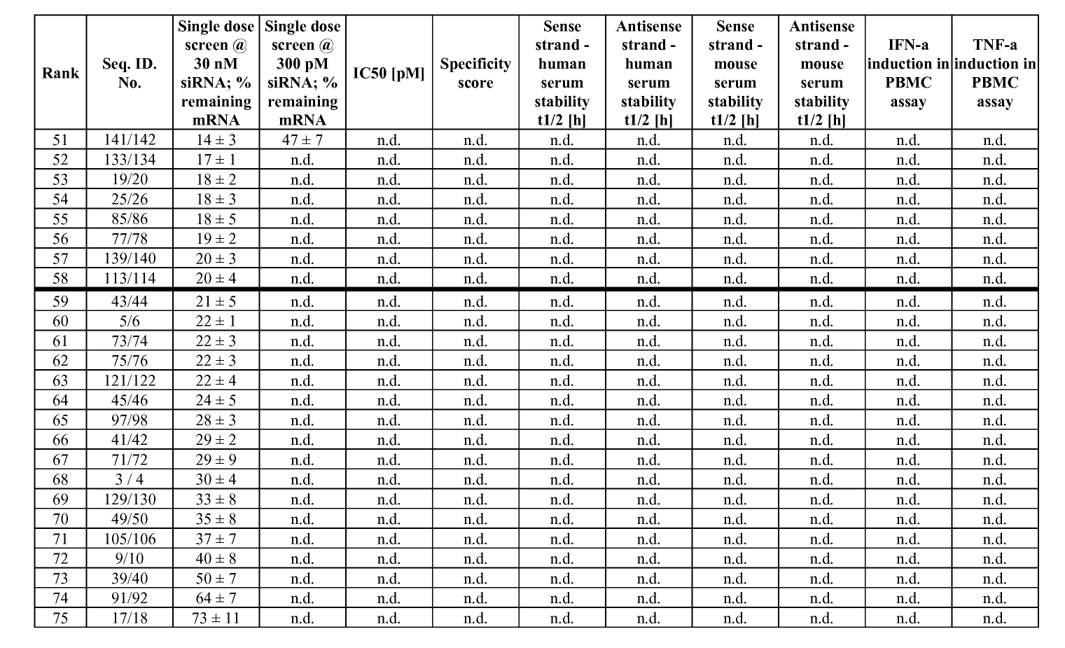

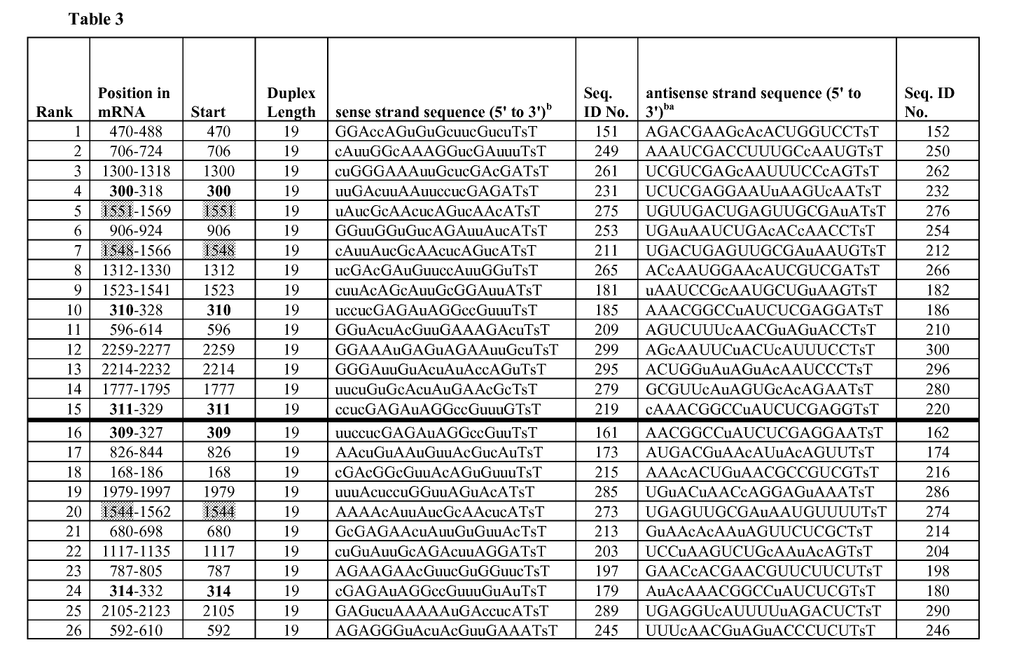

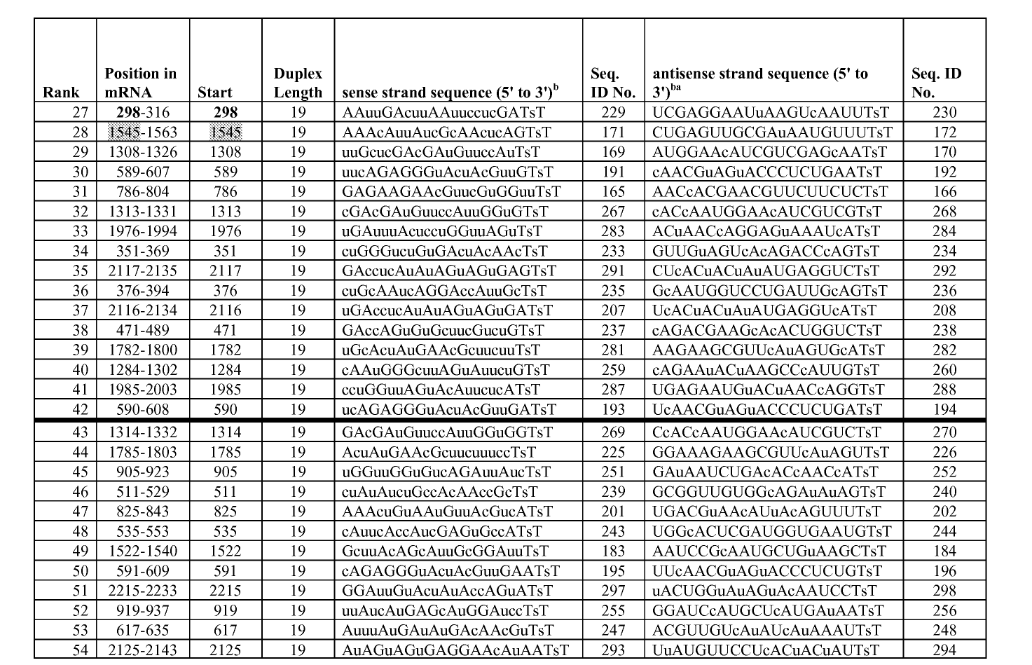

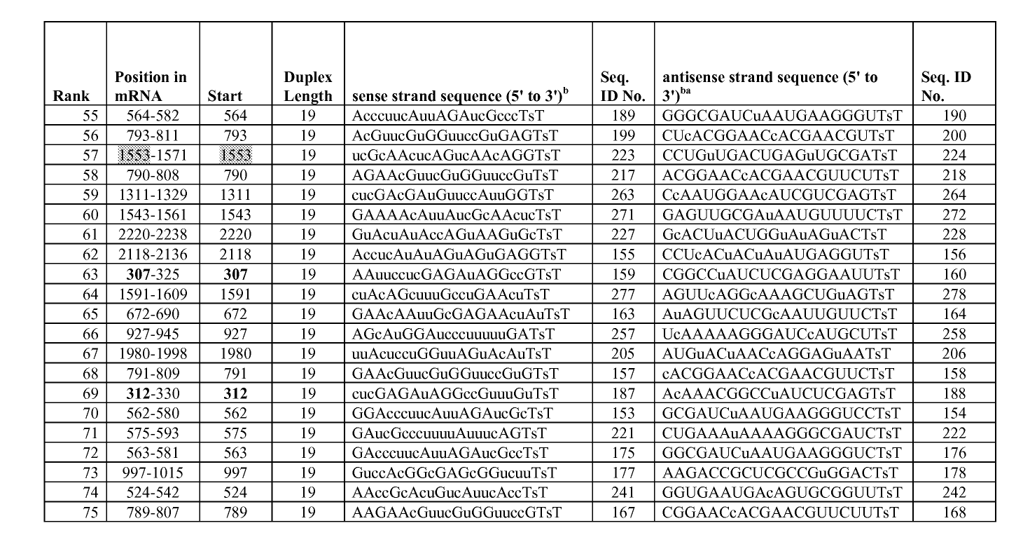

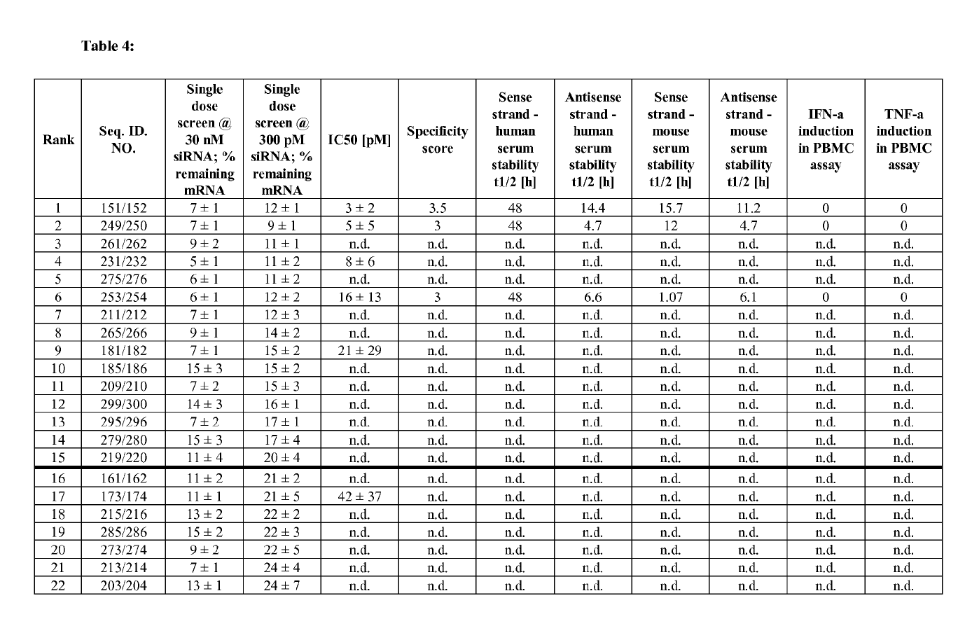

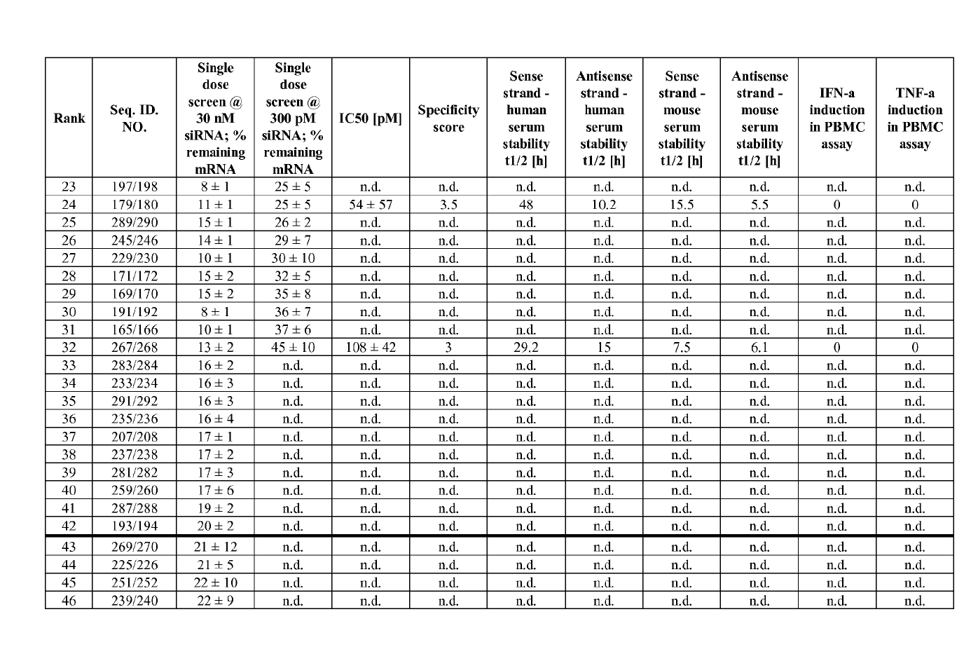

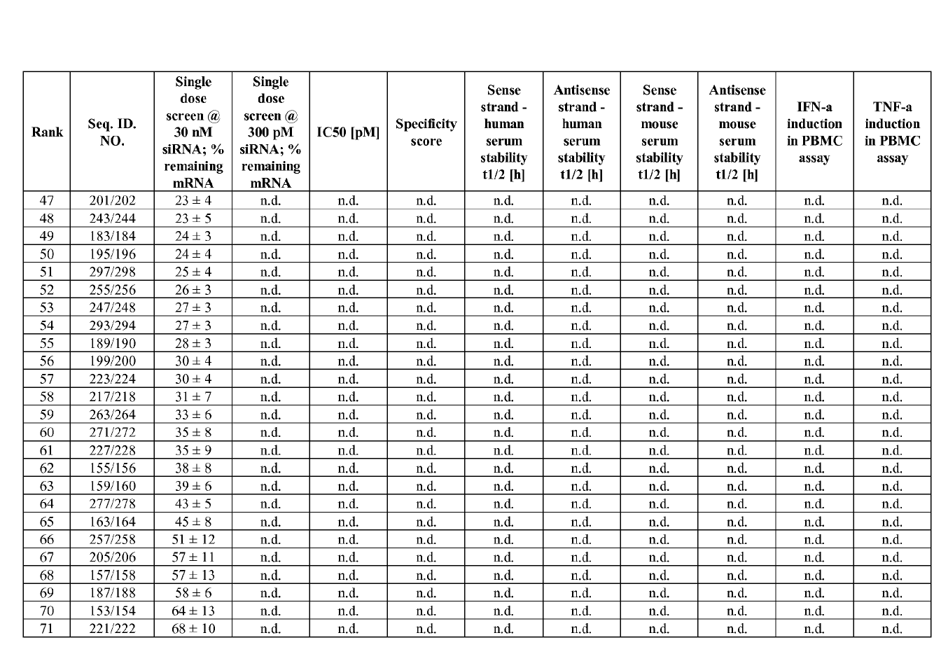

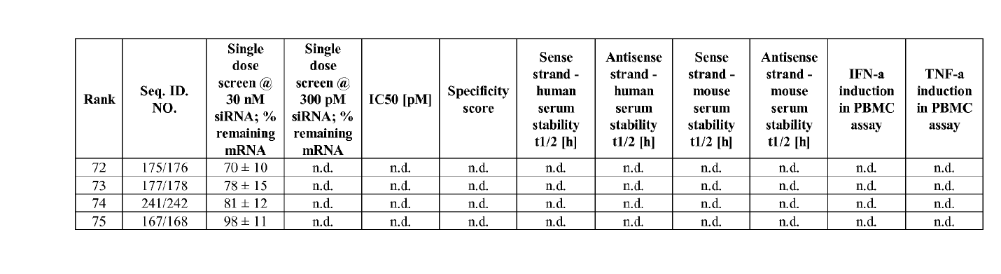

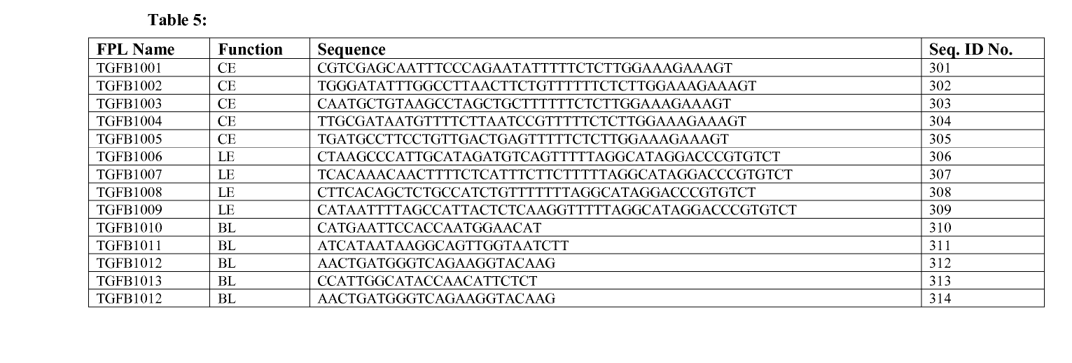

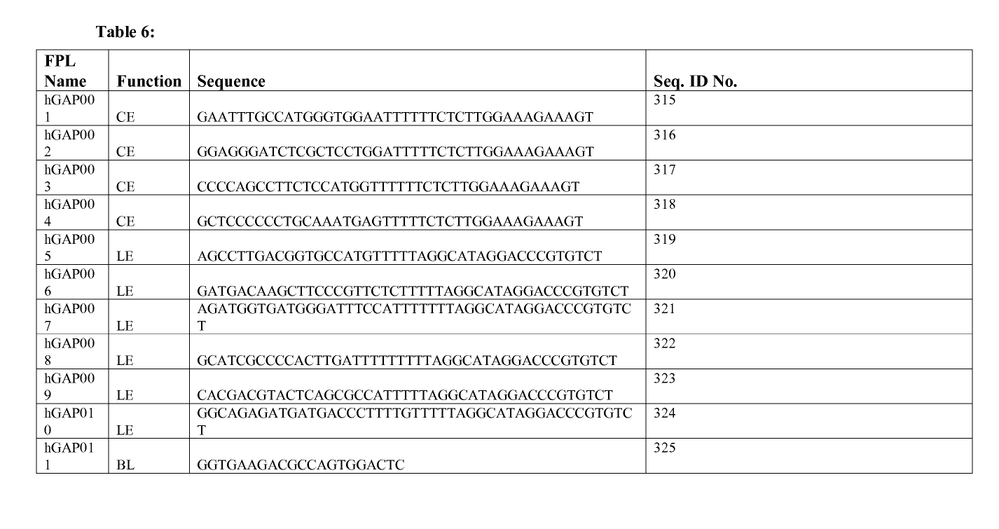

This invention relates to double-stranded ribonucleic acids (dsRNAs), and their use in mediating RNA interference to inhibit the expression of TGF-beta receptor genes, in particular in the inhibition of TGF-beta receptor type I expression. Furthermore, the use of said dsRNA to treat fibrotic diseases/disorders, inflammations and proliferative disorders, like cancers, is part of this invention.

Transforming growth factor-beta (TGF-beta; AfCS ID A002271) is part of the TGF-beta superfamily of cytokines, which has over 40 members. TGF-beta itself has at least three isoforms, including TGF-betal, TGF-beta2, and TGF-beta3. Each is a homodimer, although heterodimers can also form both between TGF-beta isoforms and other members of the TGF-beta superfamily. TGF-beta is secreted by many cell types, including macrophages, in a latent form in which it is complexed with two other polypeptides, latent TGF-beta binding protein (LTBP) and latency-associated peptide (LAP). Serum proteinases such as plasmin catalyze the release of active TGF-beta from the complex. This often occurs on the surface of macrophages where the latent TGF-beta complex is bound to CD36 via its ligand, thrombospondin-1 (TSP-1). Inflammatory stimuli that activate macrophages enhance the release of active TGF-beta by promoting the activation of plasmin. Macrophages can also endocytose IgG-bound latent TGF-beta complexes that are secreted by plasma cells and then release active TGF-beta into the extracellular fluid.

Both the type I and type II TGF-beta receptors (AfCS ID A002272/A002273) are involved in the signaling response to TGF-beta. Both are type I integral membrane proteins with a cytoplasmic serine-threonine kinase domain. Type II receptors form homodimers in the absence of ligand and can autophosphorylate each other. The type II receptors can bind TGF-beta independently of type I receptors, and they are the primary determinants of ligand specificity. The type I receptors can also form homodimers without ligand, but they do not efficiently bind ligand in the absence of type II receptors. In the presence of ligand, the type I and type II receptors form a high-avidity receptor complex. The type II receptors then phosphorylate the type I receptors, leading to their activation.

In addition to the direct activation of the Smad transcription factors (described in detail below), there is some evidence that the TGF-beta receptor can activate the ERK, JKN, and p38 MAP kinases via Ras, RhoA, and TGF-beta-activated kinase (TAK). Other reports suggest that TGF-beta receptors can signal via PI 3-kinase and protein phosphatase 2A. The mechanisms by which TGF-beta receptors activate non-Smad signaling pathways are not well understood.

Primarily, TGF-beta receptors signal via latent cytoplasmic transcription factors called Smads. The term Smad is derived from the names of the homologous Drosophila Mad proteins (short for “mothers against decapentaplegic”) and C. elegans Sma proteins (short for “small”). Upon ligand binding, the phosphorylated type I TGF-beta receptors bind and phosphorylate the receptor-regulated Smads (R-Smads), Smad1, 2, 3, 5, and 8. The binding of the R-Smads to the TGF-beta receptor complex is facilitated by a FYVE domain-containing adaptor protein called SARA and may occur after the receptor has been internalized into endosomes. Once phosphorylated, the R-Smads dissociate from the receptor complex, form homotrimers, and bind to Smad4, the common mediator Smad (Co-Smad). The R-Smad/Smad4 complex translocates into the nucleus and regulates gene transcription by interacting with tissue-specific transcriptional coactivators or corepressors. The Mad homology 1 (MH1) domains of the R-Smads and Smad4 bind 5′-AGACC-3′ Smad-binding elements (SBE).

TGF-beta receptor signaling is negatively regulated by the Smad7 inhibitory Smad (I-Smad). Complexes of Smad7 and the Smurf2 E3 ligase compete with SARA for binding to the TGF-beta receptor and promote the ubiquitination and degradation of the TGF-beta receptor complex. The Ras/ERK pathway also attenuates TGF-beta signaling to the nucleus. Phosphorylation of R-Smads by ERK prevents their nuclear accumulation.

TGF-beta has a broad range of biologic activities, too numerous to list. While it inhibits the growth of many cell types, it can also induce cell proliferation and activation. It has recently been demonstrated that the inhibition of TGF-beta receptor signaling may prevent the formation of stenosis in a rat carotid injury model (Fu et al., Arteriosclerosis, Thrombosis, and Vascular Biology 2008, 28:665). Moreover, the increased expression of the TGF-beta receptor type II seems to play an important role in the development of diabetic macroangiopathy (Hosomi et al., Atherosclerosis. 2002, 162:69-76). TGF-beta has generally been implicated in the formation of fibrotic tissues, and the inhibition of TGF-beta binding to TGF-beta receptors was shown to be capable of alleviating fibrosis (Yata et al, Hepatology 2003, 35:1022-1030).

Double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of a TGF-beta receptor gene in C. elegans.

In the liver, a major function of TGF-beta, which is normally produced by nonparenchymal stellate cells, is to limit regenerative growth of hepatocytes in response to injury by inhibiting DNA synthesis and inducing apoptosis. There is a high level of TGF-beta production in the liver of hepatocellular carcinoma (HCC) patients which may be caused by chronic hepatitis. The level of TGF-beta correlates well with HCC progression. However, TGF-beta-receptor II is downregulated in HCC cells so that they are not sensitive to TGF-beta-induced growth inhibition. Therefore the current hypothesis on the TGF-beta function in HCC is that it helps HCC cells evade immune cell attack by suppressing the immune system. HCC cells may be able to use alternative TGF-beta signaling pathways favoring growth and invasion. A TGF-beta-receptor I inhibitor has been used in preclinical studies against HCC derived liver fibrosis.

Despite significant advances in the field of RNAi and advances in the treatment of fibrosis and proliferative disorders, like cancers, there remains a need for an agent that can selectively and efficiently silence the TGF-beta receptor gene(s).

The use of RNAi is a viable pathway in the development of therapeutically active substances for the treatment of fibrotic diseases, such as, for example, hepatic fibrosis and cirrhosis, renal fibrosis, fibrosis of the spleen, cystic fibrosis of the pancreas and lungs, injection fibrosis, endomyocardial fibrosis, idiopathic pulmonary fibrosis of the lung, mediastinal fibrosis, myleofibrosis, retroperitoneal fibrosis, progressive massive fibrosis, nephrogenic systemic fibrosis, diffuse parenchymal lung disease, post-vasectomy pain syndrome, and rheumatoid arthritis. Alternatively, an inhibitor of TGF-beta receptor expression, and specifically of the expression of TGF-beta receptor I with the dsRNA molecules of this invention, may be used in the treatment of cancer, e.g. liver cancer, and, for example, HCC.

The invention provides double-stranded ribonucleic acid molecules (dsRNAs), as well as compositions and methods for inhibiting the expression of a TGF-beta receptor gene, in particular the expression of a TGF-beta receptor I gene, in a cell, tissue or mammal using such dsRNA. The invention also provides compositions and methods for treating pathological conditions and diseases caused by the expression of a TGF-beta receptor gene, in particular the TGF-beta receptor I gene, such as in fibrosis, inflammations and in proliferative disorders. The double stranded ribonucleic acid molecules of the present invention are characterized by their capability to inhibit the expression of a TGF-beta receptor I gene, in particular the mammalian and human TGF-beta receptor I gene in vitro by at least 80%. In one preferred embodiment, the inventive double-stranded ribonucleic acid molecule comprises a sense strand and an antisense strand, the antisense strand being at least partially complementary to the sense strand, whereby the sense strand comprises a sequence, which has an identity of at least 90% to at least a portion of an mRNA encoding a TGF-beta receptor, wherein said sequence is (i) located in the region of complementarity of said sense strand to said antisense strand; and (ii) wherein said sequence is less than 30 nucleotides in length.

The dsRNA of the invention comprises an RNA strand (the antisense strand) having a region which is less than 30 nucleotides in length and is substantially complementary to at least part of an mRNA transcript of a TGF-beta receptor type I gene. The use of these dsRNAs enables the targeted degradation of mRNAs of the TGF-beta receptor type I that is, inter alia, implicated in fibrosis responses, in inflammation events as well as in proliferative disorders in mammals, like in cancer for example liver cancer. Using cell-based and animal assays, the present inventors have demonstrated that very low dosages of these dsRNA can specifically and efficiently mediate RNAi, resulting in significant inhibition of expression of said TGF-beta receptor gene. Thus, the methods and compositions of the invention comprising these dsRNAs are useful for treating disorders, wherein an undesired TGF-beta receptor type I expression takes place. Such disorders comprise fibrotic disorders, inflammations as well as proliferative disorders, like cancers/tumors.

Corresponding dsRNA molecules are provided in context of this invention and most preferred dsRNA molecules are provided in the tables 1 and 3 below and, inter alia and preferably, in appended SEQ ID NOs/pairs: 1/2, 117/118, 103/104, 31/32, 81/82, 99/100, 23/24, 13/14, 29/30 and 7/8. In context of specific dsRNA molecules provided herein, pairs of SEQ ID NOs relate to corresponding sense and antisense strands sequences (5′ to 3′) as also shown in appended and included tables.

Also modified dsRNA molecules are provided herein and are in particular disclosed in table 3, providing illustrative examples of such “modified dsRNA molecules” of the present invention. Preferred molecules in this respect are, inter alia, represented by SEQ ID NOs/pairs: 151/152, 249/250, 261/262, 231/232, 275/276, 253/254, 211/212, 265/266, 181/182, 185/186, 209/210, 299/300, 295/296, 279/280 and 219/220. The illustrative modifications of these constituents of the inventive dsRNAs are provided herein as examples of modifications. Also further modifications of these dsRNAs (and their constituents) are comprised as one embodiment of this invention. Corresponding examples are provided in the more detailed description of this invention.

In one embodiment, the invention provides double-stranded ribonucleic acid (dsRNA) molecules for inhibiting the expression of a TGF-beta receptor gene, in particular the expression of the mammalian or human TGF-beta receptor type I gene. The coding sequence of the human TGF-beta receptor type I gene can be obtained from relevant databases, see, e.g. Genebank/EMBL. NM_{—}004612.2. One coding sequence which also serves as reference sequence herein for the TGF-beta receptor type I gene is provided in appended SEQ ID NO. 326.

The dsRNA comprises at least two sequences that are complementary to each other. The dsRNA comprises a sense strand comprising a first sequence and an antisense strand may comprise a second sequence, see also provision of specific dsRNA pairs in the appended tables 1 and 3. The antisense strand may comprise a nucleotide sequence which is substantially complementary to at least part of an mRNA encoding said TGF-beta receptor, and the region of complementarity is most preferably less than 30 nucleotides in length. Furthermore, it is preferred that the length of the herein described inventive dsRNA molecules (duplex length) is in the range of about 16 to 30 nucleotides, in particular in the range of about 18 to 28 nucleotides. Particularly useful in context of this invention are duplex lengths of about 19, 20, 21, 22, 23 or 24 nucleotides. Most preferred are duplex stretches of 19, 21 or 23 nucleotides. The dsRNA, upon contacting with a cell expressing a TGF-beta receptor, inhibits the expression of a TGF-beta receptor I gene in vitro by at least 80%.

Non-limiting assays how such an in vitro inhibition can be tested are provided in the appended examples, wherein activity of the siRNAs/dsRNAs of this invention and described herein was tested in HeLa, in particular in HeLaS3 cells. These HeLa cells in culture were used for quantitation of TGFbeta-receptor type I mRNA by branched DNA in total mRNA isolated from cells incubated with TGFbeta-receptor-specific siRNAs assay. This inhibition can in particular be measured in vitro. Corresponding assays can easily be established by the person skilled in the art and are also provided herein. As, e.g., shown in the appended examples or in the Tabels provided herein, the inventive dsRNAs most preferably, inhibit the expression of human TGF-beta receptor type I in vitro at a concentration of 30 nM by at least about 80%. Particular dsRNA molecules of the present invention inhibit at even lower concentration (e.g. 300 pM) in vitro the expression of the TGF-beta receptor type Ito at least about 80%. Again, corresponding exemples are provided in Tables 1 and 2, whereby in said tables, the inhibition is illustrated by the amount of remaining RNA in the assessed cells.

In one embodiment the sense strand comprises a sequence which has an identity of at least 90% to at least a portion of an mRNA encoding TGF -beta receptor type I. Said sequence is located in a region of complementarity of the sense strand to the antisense strand, preferably within nucleotides 2-7 of the 5′ terminus of the antisense strand. In one preferred embodiment the dsRNA targets particularly the human TGF-beta receptor type I gene, in yet another embodiment the dsRNA targets the mouse (Mus musculus) and rat (Rattus norvegicus) TGF-beta receptor type I gene.

In one embodiment the dsRNA molecules of the invention comprise of a sense and an antisense strand wherein both strands have a half-life of at least 5 hours. In one preferred embodiment the dsRNA molecules of the invention comprise of a sense and an antisense strand wherein both strands have a half-life of at least 5 hours in human serum.

In another embodiment the dsRNA molecules of the invention are non-immunostimulatory, e.g. do not stimulate INF-alpha and TNF-alpha in vitro.

The dsRNA molecules of the invention may be comprised of naturally occurring nucleotides or may be comprised of at least one modified nucleotide, such as a 2′-O-methyl modified nucleotide, a nucleotide comprising a 5′-phosphorothioate group, and a terminal nucleotide linked to a cholesteryl derivative or dodecanoic acid bisdecylamide group. 2′ modified nucleotides may have the additional advantage that certain immunostimulatory factors or cytokines are suppressed when the inventive dsRNA molecules are employed in vivo, for example in a medical setting. Alternatively and non-limiting, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In one preferred embodiment the dsRNA molecules comprises at least one of the following modified nucleotides: a 2′-O-methyl modified nucleotide, a nucleotide comprising a 5′-phophorothioate group and a deoxythymidine. In another preferred embodiment all pyrimidines of the sense strand are 2′-O-methyl modified nucleotides, and all pyrimidines of the antisense strand are 2′-deoxy-2′-fluoro modified nucleotides. In one preferred embodiment one of the two deoxythymidine nucleotides are found at the 3′ of both strands of the dsRNA molecule. In another embodiment at least one of these deoxythymidine nucleotides at the 3′ end of both strands of the dsRNA molecule comprises a 5′-phosphorothioate group. In another embodiment all cytosines followed by adenine, and all uracils followed by either adenine, guanine or uracil in the sense strand are 2′-O-methyl modified nucleotides, and all cytosines and uracils followed by adenine of the antisense strand are 2′-O-methyl modified nucleotides In appended Table 3 illustrative, modified double stranded RNA molecules are provided.

The dsRNA of the invention may further comprise one or more single-stranded nucleotide overhang(s). As also described above, these overhangs may in particular be at the 3′ end of the each individual strand(s) and may comprise one, two, three, four of five additional nucleotides. As also illustrated in the appended examples, of particular interest are overhangs with no, one or two additional nucleotides. In some embodiments the additional nucleotide is a “T” and preferably two “T”, i.e. an overhang with “TT” on the 3′ end of each strand.

The dsRNA molecules of the invention can be comprised of a first sequence of the dsRNA that is selected from the group consisting of the sense sequences of Table 1 or 3 and the second sequence is selected from the group consisting of the antisense sequences of Table 1 or 3. Preferred pairs of these two sequences are provided in the tables within one line/rank.

Preferably, the dsRNA comprises two oligonucleotides, wherein one oligonucleotide (sense) is described by Table 1 and the second oligonucleotide (antisense) is described Table 1 or wherein one modified oligonucleotide (sense) is described by Table 3 and the second oligonucleotide (antisense) is also described Table 3. Both Tables provide in each individual rank particular useful sense and antisense strand sequences, both provided in 5′ to 3′ direction, and these sequences in each individual rank are the preferred sequences to be used in individual dsRNAs of the present invention.

Accordingly, the first sequence of the inventive dsRNA may be selected from the group consisting of the sense sequences of Table 1 (or 3) and the second sequence may be selected from the group consisting of the antisense sequences of Table 1 (or 3), whereby Table 3 provides for exemplified 2′-O-methyl-modified sequences.

The invention also provides for cells comprising at least one of the dsRNAs of the invention. The cell is preferably a mammalian cell, such as a human cell. Furthermore, also tissues and/or non-human organisms comprising the herein defined dsRNA molecules are comprised in this invention, whereby said non-human organism is particularly useful for research purposes or as research tool, for example also in drug testing.

The invention also relates to pharmaceutical compositions comprising the inventive dsRNAs of this invention. These pharmaceutical compositions are particularly useful in the inhibition of the expression of a TGF-beta receptor type I gene in a cell, a tissue or an organism. The pharmaceutical composition comprising one or more of the dsRNA of the invention may also comprise (a) pharmaceutically acceptable carrier(s), diluent(s) and/or exipient(s). Accordingly, certain aspects of the invention provide pharmaceutical compositions comprising the dsRNA of the invention, optionally together with a pharmaceutically acceptable carrier, methods of using the compositions to inhibit expression of a TGF-beta receptor type I gene, and methods of using the pharmaceutical compositions to treat diseases caused by expression of a TGF-beta receptor gene, in particular a TGF-beta receptor type I gene.

Furthermore, the invention relates to a method for inhibiting the expression of a TGF-beta receptor gene, in particular a mammalian or human TGF-beta receptor type I gene, in a cell, tissue or organism comprising the following steps:
(a) introducing into the cell, tissue or organism a double-stranded ribonucleic acid (dsRNA) as defined herein;
(b) maintaining said cell, tissue or organism produced in step (a) for a time sufficient to obtain degradation of the mRNA transcript of a TGF-beta receptor type I gene, thereby inhibiting expression of a TGF-beta receptor type I gene in a given cell.

In another embodiment, the invention provides methods for treating, preventing or managing fibrotic disorders/diseases, inflammations or proliferative disorders, said method comprising administering to a subject in need of such treatment, prevention or management a therapeutically or prophylactically effective amount of one or more of the dsRNAs of the invention. Preferably, said subject is a mammal, most preferably a human patient.

The invention also provides for nucleic acid sequence encoding a sense strand and/or an antisense strand comprised in the double-stranded ribonucleic acid molecule as defined herein. In another embodiment, the invention provides vectors for inhibiting the expression of a TGF-beta receptor gene in a cell, in particular TGF-beta receptor type I gene comprising a regulatory sequence operable linked to a nucleotide sequence that encodes at least one strand of one of the dsRNA of the invention. Such an inventive nucleic acid molecule or vector may be comprised in a cell a tissue or a non-human organism. Such an non-human organism may be a transgenic, non-human animal. The cells, the tissues as well as the non-human transgenics of this invention may be useful as research tools. Yet, the cells and tissues may also be used in medical intervention and as pharmaceuticals.

In another embodiment, the invention provides a cell comprising a vector for inhibiting the expression of a TGF-beta receptor gene in a cell, in particular TGF-beta receptor type I gene. Said vector comprises a regulatory sequence operable linked to a nucleotide sequence that encodes at least one strand of one of the dsRNA of the invention. Yet, it is preferred that said vector comprises, besides said regulatory sequence a sequence that encodes at least one “sense strand” of the inventive dsRNA and at least one “anti sense strand” of said dsRNA. It is also envisaged that the claimed cell comprises two or more vectors comprising, besides said regulatory sequences, the herein defined sequence(s) that encode(s) at least one strand of one of the dsRNA of the invention.

The invention provides double-stranded ribonucleic acid (dsRNA), as well as compositions and methods for inhibiting the expression of a TGF-beta receptor type I gene in a cell or mammal using the dsRNA. The invention also provides compositions and methods for treating pathological conditions and diseases in a mammal caused by the expression of a TGF-beta receptor type I gene using dsRNA. dsRNA directs the sequence-specific degradation of mRNA through a process known as RNA interference (RNAi). The process occurs in a wide variety of organisms, including mammals and other vertebrates.

Selected dsRNA molecules of the present invention are provided in tables 1 and 3, whereby table 1 defines the target site in a TGFI3 receptor (type I) gene (represented also by Genebank/EMBL. NM_{—}004612.2) as well as the sense and anti-sense strand of the relevant ds RNAs. Furthermore, for certain and particularly preferred dsRNAs (sense and antisense sequences provided) biologically and clinically relevant advantageous parameters are provided; see appended table 2 and 4.

Table 1 relates also to preferred molecules to be used as dsRNA in accordance with this invention. Particularly preferred are the identified dsRNA molecules as provided in tier I (rank 1 to 10) and in tier II (rank 11 to 31). However, also tier III (rank 32 to 58), and tier IV (rank 59 to 75) comprise useful dsRNA molecules in accordance with this invention. As is evident from the above, partial preferred dsRNA molecules are provided in the sense and antisense pairs defined by SEQ ID NOs: 1/2, 117/118, 103/104, 31/32, 81/82, 99/100, 23/24, 13/14, 29/30 and/or 7/8. Table 2 provides for certain biological and clinical features of specific dsRNA molecules of the invention as shown in Table 1.

In context of the present invention, it was surprisingly be found that particular preferred dsRNAs which are useful in the inhibition of the expression of the (human) TGF-beta receptor type I gene cluster in specific regions of the corresponding mRNA of the TGF-beta receptor type I gene. In relation to the human TGF-beta receptor type I gene as provided in appended SEQ ID NO. 326 (and also in Genebank/EMBL NM_{—}004612.2), said clusters are comprised in regions of nucleotides 250 to 350 and 1500 to 1600, more preferably nucleotides 220-320 and 1520 to 1580 or more preferably in the regions of nucleotides 298-332 and 1522 to 1569 of appended SEQ ID NO. 326, representing the human TGF-beta receptor type I gene.

Tables 3 and 4 also provide for further siRNA molecules/dsRNA useful in context of this invention, whereby Table 4 provides for certain biological and/or clinically relevant surprising features of the modified siRNA molecules/dsRNA molecules of this invention as shown in Table 3. Particularly useful, modified molecules comprise the sequences (sense strand and anti-sense strand) as provided in tier I (rank 1 to 15) and tier II (rank 16 to 42). Also the dsRNA/siRNAs as defined in tier III (rank 43 to 75) comprise useful dsRNA molecules which can be employed in context of the present invention as long as an inhibition of the TGF beta receptor type I gene expression is achieved, said inhibition being measured in vitro and being an inhibition of about at least 80%. Preferred in context of modified dsRNAs/siRNAs are sequences as provided in SEQ ID NOs: 151/152, 249/250, 261/262, 231/232, 275/276, 253/254, 211/212, 265/266, 181/182, 185/186, 209/210, 299/300, 295/296, 279/280, and/or 219/220.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A”, “U” and “T” or “dT” respectively, each generally stand for a nucleotide that contains guanine, cytosine, adenine, uracil and deoxythymidine as a base, respectively. However, the term “ribonucleotide” or “nucleotide” can also refer to a modified nucleotide, as further detailed below, or a surrogate replacement moiety. Sequences comprising such replacement moieties are embodiments of the invention. As detailed below, the herein described dsRNA molecules may also comprise “overhangs”, i.e. unpaired, overhanging nucleotides which are not directly involved in the RNA double helical structure normally formed by the herein defined pair of “sense strand” and “anti sense strand”. Often, such an overhanging stretch comprises the deoxythymidine nucleotide, in most embodiments, 2 deoxythymidines in the 3′ end. Such overhangs will be described and illustrated below.

The term “TGF-beta receptor” or “transforming growth factor beta receptor” as used herein relates in particular to the TGF-beta receptor type I (TGF-beta receptor I, activin A receptor type II-like kinase) and said term relates to the corresponding gene, encoded mRNA, encoded protein/polypeptide as well as functional fragments of the same. Fragments as provides herein relate, inter alia, to the herein defined “hot spots” of clusters in the target sequence against which the herein defined dsRNA molecules are directed. Such fragments are, inter alia nucleotides 250 to 350 and 1500 to 1600 of appended SEQ ID NO. 326. The term “TGF-beta receptor type I gene/sequence” does not only relate to (the) wild-type sequence(s) but also to mutations and alterations which may be comprised in said gene/sequence. Accordingly, the present invention is not limited to the specific dsRNA molecules provided herein. The invention also relates to dsRNA molecules that comprise an antisense strand that is at least 85% complementary to the corresponding nucleotide stretch of an RNA transcript of a TGF-beta type I receptor gene that comprises such mutations/alternations.

As used herein, “target sequence” refers to a contiguous portion of the nucleotide sequence of an mRNA molecule formed during the transcription of a TGF-beta receptor Type I gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature. However, as detailed herein, such a “strand comprising a sequence” may also comprise modifications, like modified nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

Sequences referred to as “fully complementary” comprise base-pairing of the oligonucleotide or polynucleotide comprising the first nucleotide sequence to the oligonucleotide or polynucleotide comprising the second nucleotide sequence over the entire length of the first and second nucleotide sequence.

However, where a first sequence is referred to as “substantially complementary” with respect to a second sequence herein, the two sequences can be fully complementary, or they may form one or more, but preferably not more than 13 mismatched base pairs upon hybridization.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

The term “double-stranded RNA”, “dsRNA molecule”, or “dsRNA”, as used herein, refers to a ribonucleic acid molecule, or complex of ribonucleic acid molecules, having a duplex structure comprising two anti-parallel and substantially complementary nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. In addition to the duplex structure, a dsRNA may comprise one or more nucleotide overhangs. The nucleotides in said “overhangs” may comprise between 0 and 5 nucleotides, whereby “0” means no additional nucleotide(s) that form(s) an “overhang” and whereas “5” means five additional nucleotides on the individual strands of the dsRNA duplex. These optional “overhangs” are located in the 3′ end of the individual strands. As will be detailed below, also dsRNA molecules which comprise only an “overhang” in one the two strands may be useful and even advantageous in context of this invention. The “overhang” comprises preferably between 0 and 2 nucleotides. Most preferably 2 “dT” (deoxythymidine) nucleotides are found at the 3′ end of both strands of the dsRNA. Also 2 “U”(uracil) nucleotides can be used as overhangs at the 3′ end of both strands of the dsRNA. Accordingly, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. For example the antisense strand comprises 23 nucleotides and the sense strand comprises 21 nucleotides, forming a 2 nucleotide overhang at the 3′ end of the antisense strand. Preferably, the 2 nucleotide overhang is fully complementary to the mRNA of the target gene. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence. Where the region of complementarity is not fully complementary to the target sequence, the mismatches are most tolerated outside nucleotides 2-7 of the 5′ terminus of the antisense strand

The term “sense strand,” as used herein, refers to the strand of a dsRNA that includes a region that is substantially complementary to a region of the antisense strand. “Substantially complementary” means preferably at least 85% of the overlapping nucleotides in sense and antisense strand are complementary.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. It is, for example envisaged that the dsRNA molecules of this invention be administered to a subject in need of medical intervention. Such an administration may comprise the injection of the dsRNA, the vector or an cell of this invention into a diseased side in said subject, for example into liver tissue/cells or into cancerous tissues/cells, like liver cancer tissue. However, also the injection in close proximity of the diseased tissue is envisaged. In vitro introduction into a cell includes methods known in the art such as electroporation and lipofection.

The terms “silence”, “inhibit the expression of” and “knock down”, in as far as they refer to a TGF-beta receptor Type I gene, herein refer to the at least partial suppression of the expression of a TGF-beta receptor Type I gene, as manifested by a reduction of the amount of mRNA transcribed from a TGF-beta receptor Type I gene which may be isolated from a first cell or group of cells in which a TGF-beta receptor Type I gene is transcribed and which has or have been treated such that the expression of a TGF-beta receptor Type I gene is inhibited, as compared to a second cell or group of cells substantially identical to the first cell or group of cells but which has or have not been so treated (control cells). The degree of inhibition is usually expressed in terms of

$$\frac{( mRNA in controls cells ) - ( mRNA in treated cells )}{( mRNA in control cells )} \cdot 100 \%$$

Alternatively, the degree of inhibition may be given in terms of a reduction of a parameter that is functionally linked to the TGF-beta receptor Type I gene transcription, e.g. the amount of protein encoded by a TGF-beta receptor Type I gene which is secreted by a cell, or the number of cells displaying a certain phenotype.

As illustrated in the appended examples and in the appended tables provided herein, the inventive dsRNA molecules are capable of inhibiting the expression of a human TGF-beta receptor Type I gene by at least about 70%, preferably by at least 80%, most preferably by at least 90% in vitro assays, i.e in vitro. The term “in vitro” as used herein includes but is not limited to cell culture assays. In another embodiment the inventive dsRNA molecules are capable of inhibiting the expression of a mouse or rat TGF-beta receptor Type I gene by at least 70%.preferably by at least 80%, most preferably by at least 90%. The person skilled in the art can readily determine such an inhibition rate and related effects, in particular in light of the assays provided herein. As documented herein, the most preferred dsRNAs of the present invention are capable of inhibiting the expression of the human TGF-beta receptor type I gene by at least about 80% in vitro when a single dose concentration of about 30 nM of said dsRNA/siRNA is employed. Also encompassed are dsRNA/siRNA molecules that are capable of inhibiting the expression of human TGF-beta receptor type I at a single dose concentration of about 300 pM. Again, corresponding working examples in context of this invention are provided herein and are also shown in the appended tables. Particular preferred dsRNAs are provided, for example in tier I of appended Table 1, in particular in rank 1 to 31 and especially in rank 1 to 10 (sense strand and antisense strand sequences provided therein in 5′ to 3′ orientation).

The term “off target” as used herein refers to all non-target mRNAs of the transcriptome that are predicted by in silico methods to hybridize to the described dsRNAs based on sequence complementarity. The dsRNAs of the present invention preferably do specifically inhibit the expression of TGF-beta receptor Type I gene, i.e. do not inhibit the expression of any off-target.

Particular preferred dsRNAs are provided, for example in appended Table 1 and 3 (sense strand and antisense strand sequences provided therein in 5′ to 3′ orientation).

The term “half-life” as used herein is a measure of stability of a compound or molecule and can be assessed by methods known to a person skilled in the art, especially in light of the assays provided herein.

The term “non-immunostimulatory” as used herein refers to the absence of any induction of a immune response by the invented dsRNA molecules. Methods to determine immune responses are well know to a person skilled in the art, for example by assessing the release of cytokines, as described in the examples section.

The terms “treat”, “treatment”, and the like, mean in context of this invention to relief from or alleviation of a disorder related to TGF-beta receptor Type I gene expression, like fibrotic disorders, inflammations, or cancers, like liver cancer. In the context of the present invention insofar as it relates to any of the other conditions recited herein below (other than fibrosis, inflammation or cancer), the terms “treat”, “treatment”, and the like mean to relieve or alleviate at least one symptom associated with such condition, or to slow or reverse the progression of such condition.

As used herein, the phrases “therapeutically effective amount” and “prophylactically effective amount” refer to an amount that provides a therapeutic benefit in the treatment, prevention, or management of fibrosis or an overt symptom of fibrosis. The specific amount that is therapeutically effective can be readily determined by ordinary medical practitioner, and may vary depending on factors known in the art, such as, e.g. the type of fibrosis, inflammation or cancer, the patient's history and age, the stage of disease to be treated, and the administration of other medicamants, like anti-inflammatory drugs, anti-fibrosis agents or anti-cancer/anti tumor agents.

As used herein, a “pharmaceutical composition” comprises a pharmacologically effective amount of a dsRNA and a pharmaceutically acceptable carrier. However, such a “pharmaceutical composition” may also comprise the herein described vector(s) comprising a regulatory sequence operpably linked to a nucleotide sequence that encodes at least one strand of a sense or an antisense strand comprised in the inventive dsRNAs/siRNAs of this invention. It is also envisaged that cells, tissues or isolated organs that express or comprise the herein defined dsRNAs/siRNAs nay be used as “pharmaceutical compositions”, for example in medical interventions that comprise transplantation approaches. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. However, it is in particular envisaged that the pharmaceutically acceptable carrier to be employed in context of this inventions allows for the systemic adminstration of the dsRNAs, vectors or cells of this invention. Whereas also the enteric administration is envisaged the parentral administration and also transdermal or transmucosal (e.g. insufflation, buccal, vaginal , anal) administration as well was inhalation of the drug are feasible ways of administering to a patient in need of medical intervention the compounds of this invention. When parenteral administration is employed, this can comprise the direct injection of the compounds of this invention into the diseased tissue or at least in close proximity. However, also intravenous, intraarterial, subcutaneous, intramuscular, intraperitoneal, intradermal, intrathecal and other. administrations of the compounds of this invention are within the skill of the artisan, for example the attending physician.

It is in particular envisaged that the pharmaceutically acceptable carrier allows for the systemic adminstration of the dsRNAs, vectors or cells of this invention. Whereas also the enteric administration is envisaged the parentral administration and also transdermal or transmucosal (e.g. insufflation, buccal, vaginal, anal) administration as well was inhalation of the drug are feasible ways of administering to a patient in need of medical intervention the compounds of this invention. When parenteral administration is employed, this can comprise the direct injection of the compounds of this invention into the diseased tissue or at least in close proximity. However, also intravenous, intraarterial, subcutaneous, intramuscular, intraperitoneal, intradermal, intrathecal and other administrations of the compounds of this invention are within the skill of the artisan, for example the attending physician.

For intramuscular, subcutaneous and intravenous use, the pharmaceutical compositions of the invention will generally be provided in sterile aqueous solutions or suspensions, buffered to an appropriate pH and isotonicity. In a preferred embodiment, the carrier consists exclusively of an aqueous buffer. In this context, “exclusively” means no auxiliary agents or encapsulating substances are present which might affect or mediate uptake of dsRNA in the cells that express a TGF-beta receptor Type I gene. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art.

As used herein, a “transformed cell” is a cell into which at least one vector has been introduced from which a dsRNA molecule or at least one strand of such a dsRNA molecule may be expressed. Such a vector is preferably a vector comprising a regulatory sequence operably linked to nucleotide sequence that encodes at least one of a sense strand or an antisense strand comprised in the dsRNAs of this invention.

It can be reasonably expected that shorter dsRNAs comprising one of the sequences of Table 1 and Table 3 minus only a few nucleotides on one or both ends may be similarly effective as compared to the dsRNAs described above. As pointed out above, in most embodiments of this invention, the dsRNA molecules provided herein comprise a duplex length (i.e. without “overhangs”) of about 16 to about 30 nucleotides. Particular useful dsRNA duplex lengths are about 19 to about 25 nucleotides. Most preferred are duplex structures with a length of 19 nucleotides. In the inventive dsRNA molecules, the antisense strand is at least partially complementary to the sense strand.

The dsRNA of the invention can contain one or more mismatches to the target sequence. In a preferred embodiment, the dsRNA of the invention contains no more than 13 mismatches. If the antisense strand of the dsRNA contains mismatches to a target sequence, it is preferable that the area of mismatch not be located within nucleotides 2-7 of the 5′ terminus of the antisense strand. In another embodiment it is preferable that the area of mismatch not to be located within nucleotides 2-9 of the 5′ terminus of the antisense strand. Consideration of the efficacy of dsRNAs with mismatches in inhibiting expression of a TGF-beta receptor gene is important, especially if the particular region of complementarity in a TGF-beta receptor gene, in particular the TGF-beta receptor type I gene, is known to have polymorphic sequence variation within the population.

As mentioned above, at least one end/strand of the dsRNA may have a single-stranded nucleotide overhang of 1 to 5, preferably 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Preferably, the single-stranded overhang is located at the 3′-terminal end of the antisense strand or, alternatively, at the 3′-terminal end of the sense strand. The dsRNA may also have a blunt end, preferably located at the 5′-end of the antisense strand. Preferably, the antisense strand of the dsRNA has a nucleotide overhang at the 3′-end, and the 5′-end is blunt. In another embodiment, one or more of the nucleotides in the overhang is replaced with a nucleoside thiophosphate.

The dsRNA of the present invention may also be chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the dsRNA include, without limitation, methylene blue; bifunctional groups, preferably bis-(2-chloroethyl)amine; N-acetyl-N′-(p-glyoxylbenzoyl)cystamine; 4-thiouracil; and psoralen. In one preferred embodiment, the linker is a hexa-ethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is preferably formed by triple-helix bonds. Tables 3 and 4 provide examples of modified RNAi agents of the invention.

In certain embodiments, a chemical bond may be formed by means of one or several bonding groups, wherein such bonding groups are preferably poly-(oxyphosphinicooxy-1,3-propandiol)- and/or polyethylene glycol chains. In other embodiments, a chemical bond may also be formed by means of purine analogs introduced into the double-stranded structure instead of purines. In further embodiments, a chemical bond may be formed by azabenzene units introduced into the double-stranded structure. In still further embodiments, a chemical bond may be formed by branched nucleotide analogs instead of nucleotides introduced into the double-stranded structure. In certain embodiments, a chemical bond may be induced by ultraviolet light.

In yet another embodiment, the nucleotides at one or both of the two single strands may be modified to prevent or inhibit the activation of cellular enzymes, for example certain nucleases. Techniques for inhibiting the activation of cellular enzymes are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, preferably by a 2′-amino or a 2′-methyl group. Also, at least one nucleotide may be modified to form a locked nucleotide. Such locked nucleotide contains a methylene bridge that connects the 2′-oxygen of ribose with the 4′-carbon of ribose. Introduction of a locked nucleotide into an oligonucleotide improves the affinity for complementary sequences and increases the melting temperature by several degrees.

Modifications of dsRNA molecules provided herein may positively influence their stability in vivo as well as in vitro and also improve their delivery to the (diseased) target side. Furthermore, such structural and chemical modifications may positively influence physiological reactions towards the dsRNA molecules upon administration, e.g. the cytokine release which is preferably suppressed. Such chemical and structural modifications are known in the art and are, inter alia, illustrated in Nawrot (2006) Current Topics in Med Chem, 6, 913-925.

Conjugating a ligand to a dsRNA can enhance its cellular absorption as well as targeting to a particular tissue. In certain instances, a hydrophobic ligand is conjugated to the dsRNA to facilitate direct permeation of the cellular membrane. Alternatively, the ligand conjugated to the dsRNA is a substrate for receptor-mediated endocytosis. These approaches have been used to facilitate cell permeation of antisense oligonucleotides. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Attachment of folic acid to the 3′-terminus of an oligonucleotide results in increased cellular uptake of the oligonucleotide (Li, S.; Deshmukh, H. M.; Huang, L. Pharm. Res. 1998, 15, 1540). Other ligands that have been conjugated to oligonucleotides include polyethylene glycols, carbohydrate clusters, cross-linking agents, porphyrin conjugates, and delivery peptides.

In certain instances, conjugation of a cationic ligand to oligonucleotides often results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See M. Manoharan Antisense & Nucleic Acid Drug Development 2002, 12, 103 and references therein.

The ligand-conjugated dsRNA of the invention may be synthesized by the use of a dsRNA that bears a pendant reactive functionality, such as that derived from the attachment of a linking molecule onto the dsRNA. This reactive oligonucleotide may be reacted directly with commercially-available ligands, ligands that are synthesized bearing any of a variety of protecting groups, or ligands that have a linking moiety attached thereto. The methods of the invention facilitate the synthesis of ligand-conjugated dsRNA by the use of, in some preferred embodiments, nucleoside monomers that have been appropriately conjugated with ligands and that may further be attached to a solid-support material. Such ligand-nucleoside conjugates, optionally attached to a solid-support material, are prepared according to some preferred embodiments of the methods of the invention via reaction of a selected serum-binding ligand with a linking moiety located on the 5′ position of a nucleoside or oligonucleotide. In certain instances, an dsRNA bearing an aralkyl ligand attached to the 3′-terminus of the dsRNA is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The dsRNA used in the conjugates of the invention may be conveniently and routinely made through the well-known technique of solid-phase synthesis. It is also known to use similar techniques to prepare other oligonucleotides, such as the phosphorothioates and alkylated derivatives.

Teachings regarding the synthesis of particular modified oligonucleotides may be found in the following U.S. patents: U.S. Pat. No. 5,218,105, drawn to polyamine conjugated oligonucleotides; U.S. Pat. Nos. 5,541,307, drawn to oligonucleotides having modified backbones; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. No. 5,587,361 drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,608,046, both drawn to conjugated 4′-desmethyl nucleoside analogs; U.S. Pat. No. 5,610,289, drawn to backbone-modified oligonucleotide analogs; U.S. Pat. No. 6,262,241 drawn to, inter alia, methods of synthesizing 2′-fluoro-oligonucleotides.

In the ligand-conjugated dsRNA and ligand-molecule bearing sequence-specific linked nucleosides of the invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a preferred embodiment, the oligonucleotides or linked nucleosides of the invention are synthesized by an automated synthesizer using phosphoramidites derived from ligand-nucleoside conjugates in addition to commercially available phosphoramidites.

The incorporation of a 2′-O-methyl, 2′-O-ethyl, 2′-O-propyl, 2′-O-allyl, 2′-O-aminoalkyl or 2′-deoxy-2′-fluoro group in nucleosides of an oligonucleotide confers enhanced hybridization properties to the oligonucleotide. Further, oligonucleotides containing phosphorothioate backbones have enhanced nuclease stability. Thus, functionalized, linked nucleosides of the invention can be augmented to include either or both a phosphorothioate backbone or a 2′-O-methyl, 2′-O-ethyl, 2′-O-propyl, 2′-O-aminoalkyl, 2′-O-allyl or 2′-deoxy-2′-fluoro group.

In some preferred embodiments, functionalized nucleoside sequences of the invention possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In a preferred embodiment, ligand molecules may be conjugated to oligonucleotides at the 5′-position by the use of a ligand-nucleoside phosphoramidite wherein the ligand is linked to the 5′-hydroxy group directly or indirectly via a linker Such ligand-nucleoside phosphoramidites are typically used at the end of an automated synthesis procedure to provide a ligand-conjugated oligonucleotide bearing the ligand at the 5′-terminus.

In one preferred embodiment of the methods of the invention, the preparation of ligand conjugated oligonucleotides commences with the selection of appropriate precursor molecules upon which to construct the ligand molecule. Typically, the precursor is an appropriately-protected derivative of the commonly-used nucleosides. For example, the synthetic precursors for the synthesis of the ligand-conjugated oligonucleotides of the invention include, but are not limited to, 2′-aminoalkoxy-5′-ODMT-nucleosides, 2′-6-aminoalkylamino-5′-ODMT-nucleosides, 5′-6-aminoalkoxy-2′-deoxy-nucleosides, 5′-6-aminoalkoxy-2-protected-nucleosides, 3′-6-aminoalkoxy-5′-ODMT-nucleosides, and 3′-aminoalkylamino-5′-ODMT-nucleosides that may be protected in the nucleobase portion of the molecule. Methods for the synthesis of such amino-linked protected nucleoside precursors are known to those of ordinary skill in the art.

In many cases, protecting groups are used during the preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y., 1991.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821.

Additional amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the invention.

Many solid supports are commercially available and one of ordinary skill in the art can readily select a solid support to be used in the solid-phase synthesis steps. In certain embodiments, a universal support is used. A universal support allows for preparation of oligonucleotides having unusual or modified nucleotides located at the 3′-terminus of the oligonucleotide. For further details about universal supports see Scott et al., Innovations and Perspectives in solid-phase Synthesis, 3rd International Symposium, 1994, Ed. Roger Epton, Mayflower Worldwide, 115-124]. In addition, it has been reported that the oligonucleotide can be cleaved from the universal support under milder reaction conditions when oligonucleotide is bonded to the solid support via a syn-1,2-acetoxyphosphate group which more readily undergoes basic hydrolysis. See Guzaev, A. I.; Manoharan, M. J. Am. Chem. Soc. 2003, 125, 2380.

The nucleosides are linked by phosphorus-containing or non-phosphorus-containing covalent internucleoside linkages. For the purposes of identification, such conjugated nucleosides can be characterized as ligand-bearing nucleosides or ligand-nucleoside conjugates. The linked nucleosides having an aralkyl ligand conjugated to a nucleoside within their sequence will demonstrate enhanced dsRNA activity when compared to like dsRNA compounds that are not conjugated.

The aralkyl-ligand-conjugated oligonucleotides of the invention also include conjugates of oligonucleotides and linked nucleosides wherein the ligand is attached directly to the nucleoside or nucleotide without the intermediacy of a linker group. The ligand may preferably be attached, via linking groups, at a carboxyl, amino or oxo group of the ligand. Typical linking groups may be ester, amide or carbamate groups.

Specific examples of preferred modified oligonucleotides envisioned for use in the ligand-conjugated oligonucleotides of the invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes of the invention, modified oligonucleotides that do not have a phosphorus atom in their intersugar backbone can also be considered to be oligonucleosides.

Specific oligonucleotide chemical modifications are described below. It is not necessary for all positions in a given compound to be uniformly modified. Conversely, more than one modifications may be incorporated in a single dsRNA compound or even in a single nucleotide thereof.

Preferred modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included.

Representative United States Patents relating to the preparation of the above phosphorus-atom-containing linkages include, but are not limited to, U.S. Pat. Nos. 4,469,863; 5,023,243; 5,264,423; 5,321,131; 5,399,676; 5,405,939; 5,453,496; 5,455,233 and 5,466,677, each of which is herein incorporated by reference.

Preferred modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents relating to the preparation of the above oligonucleosides include, but are not limited to, U.S. Pat. Nos. 5,034,506; 5,214,134; 5,216,141; 5,264,562; 5,466,677; 5,470,967; 5,489,677; 5,602,240 and 5,663,312, each of which is herein incorporated by reference.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Teaching of PNA compounds can be found for example in U.S. Pat. No. 5,539,082.

Some preferred embodiments of the invention employ oligonucleotides with phosphorothioate linkages and oligonucleosides with heteroatom backbones, and in particular—CH₂—NH—O—CH₂ —, —CH₂—N(CH₃)—O—CH₂ — [known as a methylene (methylimino) or MMI backbone], —CH₂—O—N(CH₃)—CH₂ —, —CH₂—N(CH₃)—N(CH₃)—CH₂—, and —O—N(CH₃)—CH₂ —CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

The oligonucleotides employed in the ligand-conjugated oligonucleotides of the invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents relating to the preparation of certain of the above-noted modified nucleobases as well as other modified nucleobases include, but are not limited to, the above noted U.S. Pat. No. 3,687,808, as well as U.S. Pat. Nos. 5,134,066; 5,459,255; 5,552,540; 5,594,121 and 5,596,091 all of which are hereby incorporated by reference.

In certain embodiments, the oligonucleotides employed in the ligand-conjugated oligonucleotides of the invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. a preferred modification includes 2′-methoxyethoxy [2′-O—CH₂CH₂OCH₃, also known as 2′-O-(2-methoxyethyl) or 2′-MOE], i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, filed on Jan. 30, 1998, the contents of which are incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and, inter alia, those which are disclosed by Delgardo et. al. (Critical Reviews in Therapeutic Drug Carrier Systems 1992, 9:249), which is hereby incorporated by reference in its entirety. Further sugar modifications are disclosed by Cook (Anti-fibrosis Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230. Further representative 2′-substituent groups amenable to the invention include those having one of formula I or II:

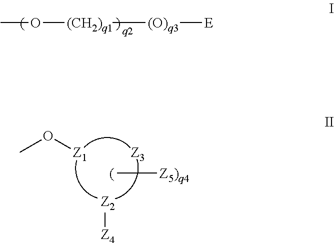

wherein,

E is C₁-C₁₀ alkyl, N(Q₃)(Q₄) or N=C (Q₃)(Q₄); each Q₃ and Q₄ is, independently, H, C₁-C₁₀ alkyl, dialkylaminoalkyl, a nitrogen protecting group, a tethered or untethered conjugate group, a linker to a solid support; or Q₃ and Q_{4,} together, form a nitrogen protecting group or a ring structure optionally including at least one additional heteroatom selected from N and O;

q₁ is an integer from 1 to 10;

q₂ is an integer from 1 to 10;

q₃ is 0 or 1;

q₄ is 0, 1 or 2;

each Z₁, Z₂ and Z₃ is, independently, C₄-C₇ cycloalkyl, C₅-C₁₄ aryl or C₃-C₁₅ heterocyclyl, wherein the heteroatom in said heterocyclyl group is selected from oxygen, nitrogen and sulfur;

Z₄ is OM₁, SM₁, or N(M₁)₂; each M₁ is, independently, H, C₁-C₈ alkyl, C₁-C₈ haloalkyl, C(=NH)N(H)M₂, C(=O)N(H)M₂ or OC(=O)N(H)M₂; M₂ is H or C₁-C₈ alkyl; and

Z₅ is C₁-C₁₀ alkyl, C₁ -C₁₀ haloalkyl, C₂-C₁₀ alkenyl, C₂-C₁₀ alkynyl, C₆-C₁₄ aryl, N(Q₃)(Q₄), OQ_{3,} halo, SQ₃ or CN.

Representative 2′-O-sugar substituent groups of formula I are disclosed in U.S. Pat. No. 6,172,209, entitled “Capped 2′-Oxyethoxy Oligonucleotides,” hereby incorporated by reference in its entirety. Representative cyclic 2′-O-sugar substituent groups of formula II are disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the invention. Representative substitutions for ring O include, but are not limited to, S, CH₂, CHF, and CF₂.

Oligonucleotides may also have sugar mimetics, such as cyclobutyl moieties, in place of the pentofuranosyl sugar. Representative United States patents relating to the preparation of such modified sugars include, but are not limited to, U.S. Pat. Nos. 5,359,044; 5,466,786; 5,519,134; 5,591,722; 5,597,909; 5,646,265 and 5,700,920, all of which are hereby incorporated by reference.

Additional modifications may also be made at other positions on the oligonucleotide, particularly the 3′ position of the sugar on the 3′ terminal nucleotide. For example, one additional modification of the ligand-conjugated oligonucleotides of the invention involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

The invention also includes compositions employing oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

In certain instances, the oligonucleotide may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate. The use of a cholesterol conjugate is particularly preferred since such a moiety can increase targeting to tissues in the liver, a site of Factor V protein production.

Alternatively, the molecule being conjugated may be converted into a building block, such as a phosphoramidite, via an alcohol group present in the molecule or by attachment of a linker bearing an alcohol group that may be phosphorylated.

Importantly, each of these approaches may be used for the synthesis of ligand conjugated oligonucleotides. Amino linked oligonucleotides may be coupled directly with ligand via the use of coupling reagents or following activation of the ligand as an NHS or pentfluorophenolate ester. Ligand phosphoramidites may be synthesized via the attachment of an aminohexanol linker to one of the carboxyl groups followed by phosphitylation of the terminal alcohol functionality. Other linkers, such as cysteamine, may also be utilized for conjugation to a chloroacetyl linker present on a synthesized oligonucleotide.

One of the major gists of the present invention is the provision of pharmaceutical compositions which comprise the dsRNA molecules of this invention. Such a pharmaceutical composition may also comprise individual strands of such a dsRNA molecule or (a) vector(s) that comprise(s) a regulatory sequence operably linked to a nucleotide sequence that encodes at least one of a sense strand or an antisense strand comprised in the dsRNA molecules of this invention. Also cells and tissues which express or comprise the herein defined dsRNA molecules may be used as pharmaceutical compositions. Such cells or tissues may in particular be useful in the transplantation approaches. These approaches may also comprise xeno transplantations.

In one embodiment, the invention provides pharmaceutical compositions comprising a dsRNA, as described herein, and a pharmaceutically acceptable carrier. The pharmaceutical composition comprising the dsRNA is useful for treating a disease or disorder associated with the expression or activity of a TGF-beta receptor type I gene, such as fibrotic disorders, cancer or inflammations.

The pharmaceutical compositions of the invention are administered in dosages sufficient to inhibit expression of a TGF-beta receptor type I gene. The present inventors have found that, because of their improved efficiency, compositions comprising the dsRNA of the invention can be administered at low dosages. A maximum dosage of 5 mg dsRNA per kilogram body weight of recipient per day is sufficient to inhibit or completely suppress expression of a TGF-beta receptor type I gene.

In general, a suitable dose of dsRNA will be in the range of 0.01 to 5.0 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. The pharmaceutical composition may be administered once daily, or the dsRNA may be administered as two, three, four, five, six or more sub-doses at appropriate intervals throughout the day or even using continuous infusion. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model.

Advances in mouse genetics have generated a number of mouse models for the study of various human diseases, such as fibrosis, cancer or inflammation. Such models are used for in vivo testing of dsRNA, as well as for determining a therapeutically effective dose.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered intraveneously.

For intramuscular, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of dsRNA in the cells that express a TGF-beta receptor gene. Such substances include, for example, micellar structures, such as liposomes or capsids, as described below. Although microinjection, lipofection, viruses, viroids, capsids, capsoids, or other auxiliary agents are required to introduce dsRNA into cell cultures, surprisingly these methods and agents are not necessary for uptake of dsRNA in vivo. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

The present invention further provides devices containing the RNAi agents of the present invention, such as devices that come into contact with the blood. Examples of devices that come into contact with blood include vascular grafts, stents, orthopedic prosthesis, cardiac prosthesis, and extracorporeal circulation systems.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs of the invention can be administered in combination with other known agents effective in treatment of fibrosis, inflammation or proliferative disorders, like cancer, in particular liver cancer. In any event, the administering physician can adjust the amount and timing of dsRNA administration on the basis of results observed using standard measures of efficacy known in the art or described herein.

The RNAi agents of the present invention can also be co-administered with suitable anti-platelet agents, including, but not limited to, fibrinogen receptor antagonists (e.g. to treat or prevent unstable angina or to prevent reocclusion after angioplasty and restenosis), anticoagulants such as aspirin, thrombolytic agents such as plasminogen activators or streptokinase to achieve synergistic effects in the treatment of various vascular pathologies, or lipid lowering agents including antihypercholesterolemics (e.g. HMG CoA reductase inhibitors such as lovastatin and simvastatin, HMG CoA synthase inhibitors, etc.) to treat or prevent atherosclerosis. For example, patients suffering from coronary artery disease, and patients subjected to angioplasty procedures, would benefit from coadministration of fibrinogen receptor antagonists and present RNAi agents.

In one embodiment, the invention provides a method for treating a subject having a pathological condition mediated by the expression of a TGF-beta receptor gene, in particular the TGF-beta receptor type I gene. Such conditions comprise disorders, such as fibrotic disorders, undesired inflammation events or proliferative disorders. In this embodiment, the dsRNA acts as a therapeutic agent for controlling the expression of a TGF-beta receptor protein. The method comprises administering a pharmaceutical composition of the invention to the patient (e.g., human), such that expression of a TGF-beta receptor gene, in particular the TGF-beta receptor type I gene, is silenced. Because of their high specificity, the dsRNAs of the invention specifically target mRNAs of a TGF-beta receptor type I gene.

The compounds of the invention are in a particular useful in those conditions where anticoagulant therapy or prophylaxis is indicated, including the following.

Compounds of the invention are useful for treating or preventing fibrotic diseases, such as, for example, hepatic fibrosis and cirrhosis, renal fibrosis, fibrosis of the spleen, cystic fibrosis of the pancreas and lungs, injection fibrosis, endomyocardial fibrosis, idiopathic pulmonary fibrosis of the lung, mediastinal fibrosis, myleofibrosis, retroperitoneal fibrosis, progressive massive fibrosis, nephrogenic systemic fibrosis, diffuse parenchymal lung disease, post-vasectomy pain syndrome, and rheumatoid arthritis. Alternatively, an inventive inhibitor of TGF-beta receptor expression, and specifically of the expression of TGF-beta receptor I, may be used in the treatment of cancer, e.g. liver cancer, and, for example, hepatocellular carcinoma HCC. Yet, also further cancers or proliferative disorders, in may be treated with the means and methods provided herein. Such proliferative disorders do not only comprise primary cancers/tumors, but also secondary tumors (i.e. tumors that develop due to metastatic events). In particularly preferred embodiments of the present invention, the tumor/cancer to be treated with the compounds of this invention is a brain, breast, lung, prostate or liver cancer.

The invention thus provides the use of an anti-TGF-beta receptor dsRNA administered to a human, particularly by intravenous administration, for the treatment of fibrosis, of undesired inflammation events and/or of unwanted cell growth.

The pharmaceutical compositions encompassed by the invention may be administered by any means known in the art including, but not limited to oral or parenteral routes, including intravenous, intramuscular, intraperitoneal, subcutaneous, transdermal, airway (aerosol), nasal, rectal, vaginal and topical (including buccal and sublingual) administration, and epidural administration. In preferred embodiments, the pharmaceutical compositions are administered intravenously by infusion or injection.

In yet another aspect, the invention provides a method for inhibiting the expression of a TGF-beta receptor type I gene in a mammal. The method comprises administering a composition of the invention to the mammal such that expression of the target TGF-beta receptor gene is silenced. Because of their high specificity, the dsRNAs of the invention specifically target RNAs (primary or processed) of the target TGF-beta receptor gene. Compositions and methods for inhibiting the expression of these TGF-beta receptor type I genes using the inventive dsRNAs can be performed as described elsewhere herein.

In another aspect of the invention, TGF-beta receptor specific dsRNA molecules that modulate TGF-beta receptor gene expression activity are expressed from transcription units inserted into DNA or RNA vectors. These transgenes can be introduced as a linear construct, a circular plasmid, or a viral vector, which can be incorporated and inherited as a transgene integrated into the host genome. The transgene can also be constructed to permit it to be inherited as an extrachromosomal plasmid.

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are preferably DNA plasmids or viral vectors. dsRNA expressing viral vectors can be constructed based on, but not limited to, adeno-associated virus; adenovirus or alphavirus as well as others known in the art. Retroviruses have been used to introduce a variety of genes into many different cell types, including epithelial cells, in vitro and/or in vivo. Recombinant retroviral vectors capable of transducing and expressing genes inserted into the genome of a cell can be produced by transfecting the recombinant retroviral genome into suitable packaging cell lines such as PA317 and Psi-CRIP. Recombinant adenoviral vectors can be used to infect a wide variety of cells and tissues in susceptible hosts (e.g., rat, hamster, dog, and chimpanzee), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or Ul snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515).

In addition, expression of the transgene can be precisely regulated, for example, by using an inducible regulatory sequence and expression systems such as a regulatory sequence that is sensitive to certain physiological regulators, e.g., circulating glucose levels, or hormones. Such inducible expression systems, suitable for the control of transgene expression in cells or in mammals include regulation by ecdysone, by estrogen, progesterone, tetracycline, chemical inducers of dimerization, and isopropyl-beta-D1 -thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single A TGF-beta receptor gene or multiple A TGF-beta receptor genes over a period of a week or more are also contemplated by the invention. Successful introduction of the vectors of the invention into host cells can be monitored using various known methods.

For example, transient transfection. can be signaled with a reporter, such as a fluorescent marker, such as Green Fluorescent Protein (GFP). Stable transfection. of ex vivo cells can be ensured using markers that provide the transfected cell with resistance to specific environmental factors (e.g., antibiotics and drugs), such as hygromycin B resistance.

In one embodiment, the method comprises administering a composition comprising a dsRNA, wherein the dsRNA comprises a nucleotide sequence which is complementary to at least a part of an RNA transcript of a TGF-beta receptor type I gene of the mammal to be treated. As pointed out above, also vectors and cells comprising nucleic acid molecules that encode for at least one strand of the herein defined dsRNA molecules can be used as pharmaceutical compositions and may, therefore, also be employed in the herein disclosed methods of treating a subject in need of medical intervention. When the organism/subject to be treated is a mammal such as a human, the composition may be administered by any means known in the art including, but not limited to oral or parenteral routes, including intravenous, intramuscular, intracranial, subcutaneous, transdermal, airway (aerosol), nasal, rectal, vaginal and topical (including buccal and sublingual) administration. In preferred embodiments, the compositions are administered by intravenous infusion or injection. Further means of administration have been, in non-limiting fashion, provided above. It is also of note that these embodiments relating to pharmaceutical compositions and to corresponding methods of treating a (human) subject also relate to approaches like gene therapy approaches. TGF-beta receptor type I specific dsRNA molecules as provided herein or nucleic acid molecules encoding individual strands of these inventive dsRNA molecules may also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Also for the introduction of dsRNA molecules, means and methods have been provided For example, targeted delivery by glycosylated and folate-modified molecules, including the use of polymeric carriers with ligands, such as galactose and lactose or the attachment of folic acid to various macromolecules allows the binding of molecules to be delivered to folate receptors. Targeted delivery by peptides and proteins other than antibodies, for example, including RGD-modified nanoparticles to deliver siRNA in vivo or multicomponent (nonviral) delivery systems including short cyclodextrins, adamantine-PEG are known. Yet, also the targeted delivery using antibodies or antibody fragments, including (monovalent) Fab-fragments of an antibody (or other fragments of such an antibody) or single-chain antibodies are envisaged. Injection approaches for target directed delivery comprise, inter alia, hydrodynamic i.v. injection. Also cholesterol conjugates of dsRNA may be used for targeted delivery, whereby the conjugation to lipohilic groups enhances cell uptake and improve pharmacokinetics and tissue biodistribution of oligonucleotides. Also cationic delivery systems are known, whereby synthetic vectors with net positive (cationic) charge to facilitate the complex formation with the polyanionic nucleic acid and interaction with the negatively charged cell membrane. Such cationic delivery systems comprise also cationic liposomal delivery systems, cationic polymer and peptide delivery systems. Other delivery systems for the cellular uptake of dsRNA/siRNA are aptamer-ds/siRNA. Also gene therapy approaches can be used to deliver the inventive dsRNA molecules or nucleic acid molecules encoding the same. Such systems comprise the use of non-pathogenic virus, modified viral vectors, as well as deliveries with nanoparticles or liposomes. Other delivery methods for the cellular uptake of dsRNA are extracorporeal, for example ex vivo treatments of cells, organs or tissues. Certain of these technologies are described and summarized in publications, like Akhtar (2007), Journal of Clinical Investigation 117, 3623-3632, Nguyen et al. (2008), Current Opinion in Moleculare Therapeutics 10, 158-167, Zamboni (2005), Clin Cancer Res 11, 8230-8234 or Ikeda et al. (2006), Pharmaceutical Research 23, 1631-1640.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items of the present invention are now illustrated with the following, non-limiting examples.

Description of appended tables:

Table 1—dsRNA targeting human TGF-beta receptor I gene. First number in column “position in mRNA” corresponds to the start of the 23 mer sequence. Numbers in said column marked in grey or bold indicate a hotspot (grey=hotspot 1; bold=hotspot 2). Length of the duplex is for all sequences 19 nucleotides.

Table 2—Characterization of dsRNAs targeting human TGF-beta receptor I: Activity testing for single dose and dose response in HeLaS3 cells, specifity, stability and Cytokine Induction. IC 50: 50% inhibitory concentration.t ½: half-life of a strand as defined in examples, PBMC: Human peripheral blood mononuclear cells.

Table 3—dsRNA targeting human TGF-beta receptor I gene comprising nucleotide modifications. Numbers in column “position in mRNA” marked in grey or bold indicate a hotspot (grey=hotspot 1; bold=hotspot 2). Letters in capitals represent RNA nucleotides, lower case letters “c”, “g”, “a” and “u” represent 2′O-methyl-modified nucleotides, “s” represents phosphorothioate.

Table 4—Characterization of dsRNAs targeting human TGF-beta receptor I comprising nucleotide modifications: Activity testing for single dose and dose response in HeLaS3 cells, specifity, stability and Cytokine Induction. IC 50: 50% inhibitory concentration.t ½: half-life of a strand as defined in examples, PBMC: Human peripheral blood mononuclear cells.

Table 5—Sequences of bDNA probes for determination of human TGF-beta receptor I; LE=label extender, CE=capture extender, BL=blocking probe.

Table 6—Sequences of bDNA probes for determination of human GAPDH; LE=label extender, CE=capture extender, BL=blocking probe.

### EXAMPLES

Gene Walking of a TGF-beta Receptor Gene

siRNA design was carried out to identify siRNAs targeting human TGF-beta receptor I. First, the known mRNA sequences of Homo sapiens TGF-beta receptor I (NM_{—}004612.2, L11695.1) were examined by computer analysis to identify homologous sequences of 19 nucleotides that yield RNAi agents cross-reactive between these sequences.

In identifying RNAi agents, the selection was limited to 19 mer sequences having at least 2 mismatches to any other sequence in the human RefSeq database (release 24), which we assumed to represent the comprehensive human transcriptome, by using the fastA algorithm.

The sequences thus identified formed the basis for the synthesis of the RNAi agents in Table 1 and Table 3.

dsRNA Synthesis

Source of Reagents

Where the source of a reagent is not specifically given herein, such reagent may be obtained from any supplier of reagents for molecular biology at a quality/purity standard for application in molecular biology.

siRNA Synthesis

Single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleil3heim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

Activity Testing

The activity of the siRNAs described above was tested in HeLaS3 cells.

HeLa cells in culture were used for quantitation of TGFbeta-receptor type I mRNA by branched DNA in total mRNA isolated from cells incubated with TGFbeta-receptor-specific siRNAs assay.

HeLaS3 cells were obtained from American Type Culture Collection (Rockville, Md., cat. No. CCL-2.2) and cultured in Ham's F12 (Biochrom AG, Berlin, Germany, cat. No. FG 0815) supplemented to contain 10% fetal calf serum (FCS) (Biochrom AG, Berlin, Germany, cat. No. S0115), Penicillin 100 U/ml, Streptomycin 100 mg/ml (Biochrom AG, Berlin, Germany, cat. No. A2213) at 37° C. in an atmosphere with 5% CO2 in a humidified incubator (Heraeus HERAcell, Kendro Laboratory Products, Langenselbold, Germany). Cell seeding and transfection of siRNA were performed at the same time. For transfection with siRNA, HeLaS3 cells were seeded at a density of 1.5.times.10.sup.4 cells/well in 96-well plates. Transfection of siRNA was carried out with lipofectamine 2000 (Invitrogen GmbH, Karlsruhe, Germany, cat. No. 11668-019) as described by the manufacturer. In a first single dose experiment siRNAs were transfected at a concentration of 30 nM. In a second single dose experiment most active siRNAs were reanalyzed at 300 pM. Most effective siRNAs against TGFbeta-receptor from the single dose screen at 300 pM were further characterized by dose response curves. For dose response curves, transfections were performed as for the single dose screen above, but with the following concentrations of siRNA (nM): 24, 6, 1.5, 0.375, 0.0938, 0.0234, 0.0059, 0.0015, 0.0004 and 0.0001 nM . After transfection cells were incubated for 24 h at 37° C. and 5% CO2 in a humidified incubator (Heraeus GmbH, Hanau, Germany). For measurement of TGFbeta-receptor mRNA cells were harvested and lysed at 53° C. following procedures recommended by the manufacturer of the QuantiGene Screen Assay Kit (Cat-No: QG0004, Panomics, Inc., Fremont, USA) for bDNA quantitation of mRNA. Afterwards, 50 μl of the lysates were incubated with probesets specific to human TGFbeta-receptor and human GAPDH (sequence of probesets see appended tables 5 and 6) and processed according to the manufacturer's protocol for QuantiGene. Chemoluminescence was measured in a Victor2-Light (Perkin Elmer, Wiesbaden, Germany) as RLUs (relative light units) and values obtained with the human TGFbeta-receptor probeset were normalized to the respective human GAPDH values for each well. Unrelated control siRNAs were used as a negative control.

Stability of SiRNAs

Stability of siRNAs was determined in in vitro assays with either human or mouse serum by measuring the half-life of each single strand.

Measurements were carried out in triplicates for each time point, using 3 μl 50 μM siRNA sample mixed with 30 μl human or mouse serum (Sigma Aldrich). Mixtures were incubated for either 0 min, 30 min, 1 h, 3 h, 6 h, 24 h, or 48 h at 37° C. As control for unspecific degradation siRNA was incubated with 3 μl 1×PBS pH 6.8 for 48 h. Reactions were stopped by the addition of 4 μl proteinase K (20 mg/ml), 25 μl of proteinase K buffer and 33 μl Millipore water for 20 min at 65° C. Samples were afterwards spin filtered through a 0.2 μm 96 well filter plate at 3000 rpm for 20 min, washed with 50 μl Millipore water twice and spin filtered again.

For separation of single strands and analysis of remaining full length product (FLP), samples were run through an ion exchange Dionex Summit HPLC under denaturing conditions using as eluent A 20 mM Na₃PO₄ in 10% ACN pH=11 and for eluent B 1 M NaBr in eluent A. The following gradient was applied:

| Time | % A | % B |
|---|---|---|
| −1.0 min | 75 | 25 |
| 1.00 min | 75 | 25 |
| 19.0 min | 38 | 62 |
| 19.5 min | 0 | 100 |
| 21.5 min | 0 | 100 |
| 22.0 min | 75 | 25 |
| 25.0 min | 75 | 25 |

For every injection, the chromatograms were integrated automatically by the Dionex Chromeleon 6.60 HPLC software, and were adjusted manually if necessary. All peak areas were corrected to the internal standard (IS) peak and normalized to the incubation at t=0 min. The area under the peak and resulting remaining FLP was calculated for each single strand and triplicate separately. Half-life (t½) of a strand was defined by the average time point [h] for triplicates at which half of the FLP was degraded.

Cytokine Induction

Potential cytokine induction of siRNAs was determined by measuring the release of INF-a and TNF-a in an in vitro PBMC assay.

Human peripheral blood mononuclear cells (PBMC) were isolated from buffy coat blood of two donors by Ficoll centrifugation at the day of transfection. Cells were transfected in quadruplicates with siRNA for 24 h at 37° C. at a final concentration of 130 nM in Opti-MEM, using either Gene Porter 2 (GP2) or DOTAP. siRNA sequences that were known to induce INF-a and TNF-a the assay, as well as a CpG oligo were used as positive controls at a concentration of 500 nM.

INF-a and TNF-a was measured in supernatant of pooled quadruplicates twice each by sandwich ELISA. Degree of induction was expressed relative to positive controls as score with a maximum of 5.

Specificity of SiRNAs

Specificity of siRNAs was determined by in silico prediction of its off-targeting potential.

Off-targeting potential was measured in relation to the most relevant off-target gene and expressed by a numeric specificity score. The most relevant off-target gene was identified based on mismatch number and distribution to the antisense strand of the siRNA. In order to determine all potential off-target genes, all human transcripts (RefSeq database, release 24), were searched for potential target regions with highest complementarity to the antisense sequence using fastA algorithm.

To identify the most relevant off-target gene characterized by the lowest specificity score, fastA output files were analyzed further by perl scripts. High specificity scores were defined as most favorable, with a score of at least 3 qualifying as specific.

1-20. (canceled)

21. A double-stranded ribonucleic acid molecule capable of inhibiting the expression of a human TGF-beta receptor type I gene in vitro by at least 80%.

22. A double-stranded ribonucleic acid molecule of claim 21 wherein the double-stranded ribonucleic acid molecule is less than 30 nucleotides in length.

23. A double-stranded ribonucleic acid molecule of claim 21, wherein said sense strand or antisense strand contains at least one modified nucleotide.

24. A double-stranded ribonucleic acid molecule of claim 23, wherein said modified nucleotide is selected from the group consisting of a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

25. A double-stranded ribonucleic acid molecule comprising a sense strand sequence selected from the group consisting of SEQ ID NOS: 1, 117, 103, 31, 81, 99, 23, 13, 29 and 7; and an antisense strand sequence selected from the group consisting of SEQ ID NOS: 2, 118, 104, 32, 82, 100, 24, 14, 30 and 8.

26. A double-stranded ribonucleic acid molecule of claim 25 comprising a sense/antisense sequence pair selected from the group consisting of SEQ ID NOS: 1/2 and 117/118.

27. A double-stranded ribonucleic acid molecule of claim 25 comprising a sense/antisense sequence pair selected from the group consisting of SEQ ID NOS: 103/104 and 31/32.

28. A double-stranded ribonucleic acid molecule of claim 25 comprising a sense/antisense sequence pair selected from the group consisting of SEQ ID NOS: 81/82 and 99/100.

29. A double-stranded ribonucleic acid molecule of claim 25 comprising a sense/antisense sequence pair selected from the group consisting of SEQ ID NOS: 23/24 and 13/14.

30. A double-stranded ribonucleic acid molecule of claim 25 comprising a sense/antisense sequence pair selected from the group consisting of SEQ ID NOS: 29/30 and 7/8.

31. A double-stranded ribonucleic acid molecule comprising a sense strand sequence selected from the group consisting of SEQ ID NOS: 151, 249, 261, 231, 275, 253, 211, 265, 181, 185, 209, 299, 295, 279 and 219; and an antisense strand sequence selected from the group consisting of SEQ ID NOS: 152, 250, 262, 232, 276, 254, 212, 266, 182, 186, 210, 300, 296, 280 and 220.

32. A double-stranded ribonucleic acid molecule of claim 31 comprising a sense/antisense sequence pair selected from the group consisting of SEQ ID NOS: 151/152 and 249/250.

33. A double-stranded ribonucleic acid molecule of claim 31 comprising a sense/antisense sequence pair selected from the group consisting of SEQ ID NOS: 261/262, 231/232, and 275/276.

34. A double-stranded ribonucleic acid molecule of claim 31 comprising a sense/antisense sequence pair selected from the group consisting of SEQ ID NOS: 253/254, 211/212, and 265/266.

35. A double-stranded ribonucleic acid molecule of claim 31 comprising a sense/antisense sequence pair selected from the group consisting of SEQ ID NOS: 181/182, 185/186, and 209/210.

36. A double-stranded ribonucleic acid molecule of claim 31 comprising a sense/antisense sequence pair selected from the group consisting of SEQ ID NOS: 299/300, 295/296, 279/280 and 219/220.

37. A cell, tissue or non-human organism comprising the double-stranded ribonucleic acid molecule as defined in claim 25.

38. A cell, tissue or non-human organism comprising the double-stranded ribonucleic acid molecule as defined in claim 31.

39. A pharmaceutical composition comprising the double-stranded ribonucleic acid molecule as defined in claim 25 and a pharmaceutically acceptable carrier, stabilizer or diluent.

40. A pharmaceutical composition comprising the double-stranded ribonucleic acid molecule as defined in claim 31 and a pharmaceutically acceptable carrier, stabilizer or diluent.
